(19) United States
(12) Patent Application Publication
(10) Pub. No.: US 2020/0093635 A1
(43) Pub. Date: Mar. 26, 2020

(54) METHOD AND APPARATUS FOR FACILITATING REDUCTION IN USER'S FATIGUE

(71) Applicant: DENSO CORPORATION, Kariya-city (JP)

(72) Inventors: Masaru KAKIZAKI, Kariya-city (JP); Yuki SHIMIZU, Kariya-city (JP)

(21) Appl. No.: 16/574,375

(22) Filed: Sep. 18, 2019

(30) Foreign Application Priority Data
Sep. 20, 2018 (JP) 2018-175757

(51) Int. Cl. A61F 7/00

(52) U.S. Cl. CPC A61F 7/00; A61F 2007/0023; A61F 2007/006; B60N 2/5621; A61F 7/0053; A61F 2007/0086; A61F 2007/0064

(57) ABSTRACT

In an apparatus for facilitating fatigue reduction of a user, a characteristic identifier identifies at least one personal characteristic item of the user, and a hot-cold stimulus controller instructs a hot-cold stimulator to apply hot and cold stimuluses to the user in accordance with a stimulus condition, and varies, based on the identified at least one personal characteristic item of the user, the stimulus condition of each of the hot stimulus and cold stimulus. The stimulus condition of each of the hot stimulus and cold stimulus include at least one of, one or more parts of the user to which the corresponding one of the hot stimulus and cold stimulus is to be applied, a temperature of the corresponding one of the hot stimulus and cold stimulus, and an application period of the corresponding one of the hot stimulus and cold stimulus to the user.

### CROSS-REFERENCE TO RELATED APPLICATION

This application is based on and claims the benefit of priority from earlier Japanese Patent Application No. 2018-175757 filed on Sep. 20, 2018, the description of which is incorporated herein by reference.

### TECHNICAL FIELD

The present invention relates to methods and apparatuses for facilitating, i.e. assisting, reduction in fatigue of a user, and also relates to computer-readable storage mediums each storing a set of computer program instructions, which cause a computer to control one or more devices for facilitating reduction in fatigue of a user.

### BACKGROUND

For reducing fatigue of a user, known technologies alternately warm, i.e. hot, and cool a predetermined portion of a user to thereby facilitate the blood flow of the user.

### SUMMARY

According to an exemplary aspect of the present disclosure, there is provided an apparatus for facilitating fatigue reduction of a user. The apparatus is configured to vary, based on an identified at least one personal characteristic item of a user, a stimulus condition of each of a hot stimulus and a cold stimulus. The stimulus condition of each of the hot stimulus and cold stimulus including at least one of

1. One or more parts of the user to which the corresponding one of the hot stimulus and cold stimulus is to be applied

2. A temperature of the corresponding one of the hot stimulus and cold stimulus

3. An application period of the corresponding one of the hot stimulus and cold stimulus to the user

### BRIEF DESCRIPTION OF THE DRAWINGS

Other aspects of the present disclosure will become apparent from the following description of embodiments with reference to the accompanying drawings in which:

FIG. 1 is a block diagram schematically illustrating an example of the configuration of a fatigue reduction facilitating system according to the first embodiment of the present disclosure;

FIG. 2 is an enlarged view schematically illustrating an example of how a hot-cold stimulator illustrated in FIG. 1 is installed in a driver's seat of a vehicle;

FIG. 3 is a block diagram schematically illustrating an example of the configuration of a hot-cold stimulation module illustrated in FIG. 1:

FIG. 4 is a block diagram schematically illustrating an example of the configuration of a human machine interface control unit illustrated in FIG. 1;

FIG. 5 is a timing chart schematically illustrating how the hot-cold stimulator sequentially performs a cycle of alternate hot and cold stimuli;

FIG. 6A is a diagram schematically illustrating a table storing default setting values used for a default stimulus mode;

Each of FIGS. 6B to 6F is a diagram schematically illustrating variation values with respect to the corresponding respective default setting values;

Each of FIGS. 7A and 7B is a flowchart schematically illustrating a hot-cold stimulus routine carried out by a hot-cold stimulus controller illustrated in FIG. 4;

FIG. 8 is a timing chart schematically illustrating how the hot-cold stimulator sequentially performs a cycle of alternate hot and cold stimuli while disabling cold stimuli to a waist of a user; and

FIG. 9 is an enlarged view schematically illustrating how a hot-cold stimulator according to a modification of a second embodiment of the present disclosure is installed in a driver's seat of a vehicle.

### DETAILED DESCRIPTION OF EMBODIMENT

### View Point of Disclosing Party

For reducing fatigue of a user, known technologies alternately warm, i.e. hot, and cool a predetermined portion of a user to thereby facilitate the blood flow of the user; one of these technologies is disclosed in Japanese Patent Publication No. 6094964, which will be referred to as a published patent document.

The published patent document discloses such a technology that uses a pair of hot-cool units and a controller controllably connected to the hot-cool units. Each hot-cool unit is designed to be mountable to a desired portion, such as a lower leg, of a user. The controller drives each hot-cool unit to periodically perform alternate heating and cooling of the hot-cool unit, thus periodically applying alternate hot and cold stimuli to the corresponding desired portion. This facilitates the blood flow of the user, thus contributing to reduction in fatigue of the user.

In particular, the technology also measures how the blood is flowing in the body of a user in real time, and delays the driving timing of one of the hot-cool units relative to the other thereof in accordance with the measured information about how the blood is flowing in the body of the user.

Unfortunately, the above technology disclosed in the published patent document fails to disclose countermeasures against individual discomforts of various users due to hot stimuli and/or cold stimuli to the various users. This may result in difficulty in reduction of the user's discomforts due to hot stimuli and/or cold stimuli to the various users.

From this viewpoint, the present disclosure seeks to provide methods and apparatuses for facilitating reduction in fatigue of a user, each of which is capable of reducing a discomfort of the user due to a hot stimulus and/or a cold stimulus to the user.

According to a first exemplary aspect of the present disclosure, there is provided an apparatus for facilitating fatigue reduction of a user. The apparatus includes a characteristic identifier configured to identify at least one personal characteristic item of the user, and a hot-cold stimulus controller. The hot-cold stimulus controller is configured to instruct a hot-cold stimulator to apply a hot stimulus and a cold stimulus to the user in accordance with a stimulus condition, and varies, based on the identified at least one personal characteristic item of the user, the stimulus condition of each of the hot stimulus and cold stimulus. The stimulus condition of each of the hot stimulus and cold stimulus including at least one of

1. One or more parts of the user to which the corresponding one of the hot stimulus and cold stimulus is to be applied

2. A temperature of the corresponding one of the hot stimulus and cold stimulus

3. An application period of the corresponding one of the hot stimulus and cold stimulus to the user

The at least one personal characteristic item of the user is estimated to have a high correlation with the sensitivity of the user to the hot/cold stimuli. For this reason, for the hot stimulus, where the hot stimulus is applied to, the temperature of the hot stimulus, and the application period of the hot stimulus to the user may lead to an increase of discomfort of the user having the at least one personal characteristic item. Similarly, for the cold stimulus, where the cold stimulus is applied to, the temperature of the cold stimulus, and the application period of the cold stimulus to the user may lead to an increase of discomfort of the user having the at least one personal characteristic item.

From this viewpoint, the hot-cold stimulus controller is configured to vary, based on the identified at least one personal characteristic item of the user, the stimulus condition of each of the hot stimulus and cold stimulus. The stimulus condition of each of the hot stimulus and cold stimulus including at least one of

1. One or more parts of the user to which the corresponding one of the hot stimulus and cold stimulus is to be applied

2. A temperature of the corresponding one of the hot stimulus and cold stimulus

3. An application period of the corresponding one of the hot stimulus and cold stimulus to the user

Variation of the stimulus condition of each of the hot stimulus and cold stimulus makes it possible to prevent an increase in the driver's discomfort while reducing the driver's fatigue, resulting in reduction of a user's uncomfortable feeling due to application of the hot/cold stimuli to the user for reducing fatigue of the user.

### EMBODIMENT

From the above viewpoint of the disclosing party, the following describes fatigue reduction facilitating systems, which are embodiments of the present disclosure, with reference to the accompanying drawings. In the embodiments, similar or equivalent parts between the embodiments, to which like reference characters are assigned, are omitted or simplified to avoid redundant description.

### First Embodiment

### Schematic Configuration of Fatigue Reduction Facilitating System 1

The following describes a fatigue reduction facilitating system 1 according to the first embodiment of the present disclosure.

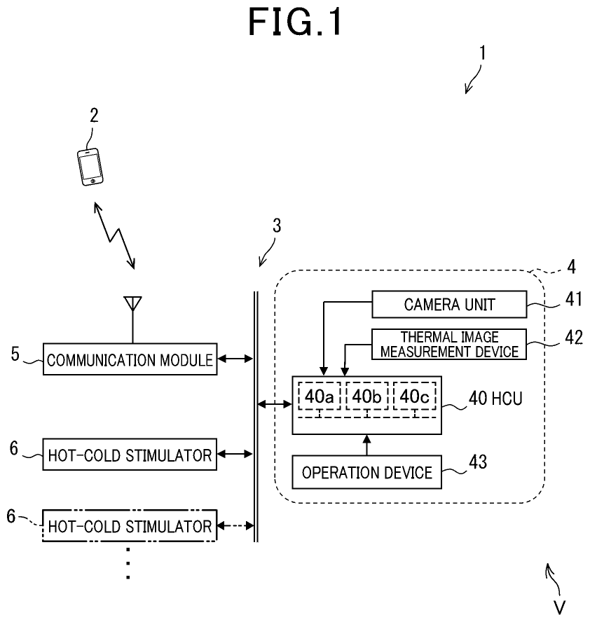

The fatigue reduction facilitating system 1 illustrated in FIG. 1 includes a mobile terminal 2 and a vehicular system 3, which is for example used in, i.e. installed in, a vehicle, such as an automobile, V. The mobile terminal 2 is carried by, for example, a driver of the vehicle V.

The mobile terminal 2 can include any carriable terminal that enables

(1) Specific communications with another system, device, or terminal to be carried out

(2) Any information to be inputtable thereto

For example, the first embodiment can use, as the mobile terminal 2, a multifunctional mobile phone, a tablet terminal, or a laptop computer.

The following describes a multifunctional mobile phone, such as a smartphone, used as the mobile terminal 2; the multifunctional mobile phone has a communication function that enables the mobile terminal 2 to communicate with another device or terminal in accordance with near field communications standards, such as Bluetooth® Low Energy standards.

### Schematic Configuration of Vehicular System 3

The following describes an example of the schematic configuration of the vehicular system 3.

Referring to FIG. 1, the vehicular system 3 includes a human machine interface (HMI) system 4, a communication module 5, and a hot-cold stimulator 6. The HMI system 4, communication module 5, and hot-cold stimulator 6 are communicably connected to each other via a communication network, such as an in-vehicle local area network installed in the vehicle V.

The HMI system 4 is provided in the interior of the vehicle V, and is comprised of a human machine interface control unit (HCU) 40, a camera unit 41, a thermal image measurement device 42, and an operation device 43. The HMI system 4 is capable of receiving information inputted from a driver of the vehicle V, and monitoring the state of a driver of the vehicle V. A driver of the vehicle V serves as, for example, a target person.

The camera unit 41, the thermal image measurement device 42, and the operation device 43 are communicably connected to the HCU 40.

The camera unit 41 is comprised of, for example, a near-infrared camera 41a and a near-infrared light source 41b. The near-infrared camera 41a is sensitive to the near-infrared range of the electromagnetic spectrum.

That is, the near-infrared light source 41b is configured to irradiate a predetermined irradiation region with near-infrared light.

The near-infrared camera 41a is comprised of, for example, a near-infrared light receiving area comprised of near-infrared light sensitive elements, i.e. pixels, that are two-dimensionally arranged in both vertical and horizontal directions corresponding to the respective height and width directions of the vehicle V.

The near-infrared camera 41a is configured to for example periodically capture an image, i.e. the assembly of light components received by the respective pixels, of a predetermined imaging region set to include the irradiation region irradiated by the near-infrared light to thereby output, to the HCU 40, the periodically captured images.

Note that the first embodiment can use, as the near-infrared light source 41b, a light emitting diode (LED) or one of other light sources, an example of which is a light source including filaments.

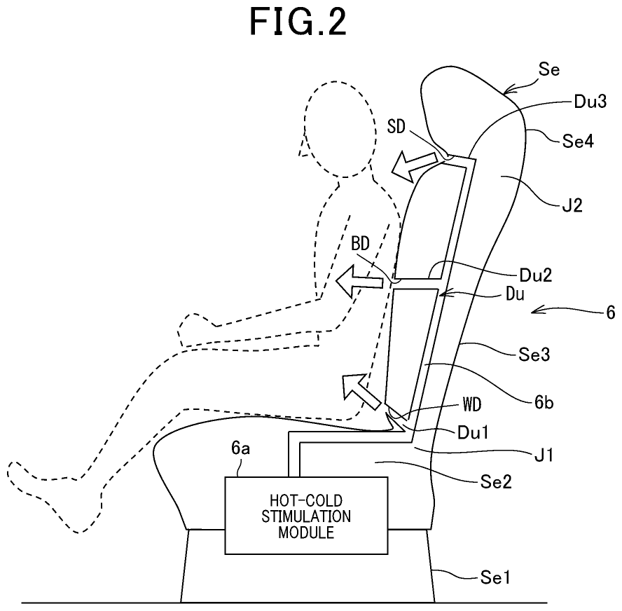

Specifically, the irradiation region is set to include the face of a driver (see dashed line DR in FIG. 2), who is sitting on a driver's seat Se (see FIG. 2) of the vehicle V, and its surrounding region, so that the near-infrared camera 41a is configured to periodically capture an image of the predetermined imaging region that is set to include the face of the driver irradiated by the near-infrared light to thereby output, to the HCU 40, the periodically captured images.

More specifically, the irradiation region according to the first embodiment is set to a region including an upper half of the body of a driver sitting on the driver's seat Se, which includes the face of the driver, so that the near-infrared camera 41a is configured to periodically capture an image of the predetermined imaging region set to include the upper half of the body of the driver irradiated by the near-infrared light to thereby output, to the HCU 40, the periodically captured images. These captured images are called “driver images” hereinafter.

For example, the near-infrared camera 41a can be mounted to the top surface of the instrumental panel of the vehicle V. Where the near-infrared camera 41a is mounted is not limited to the top surface of the instrumental panel and can be mounted to another portion in the vehicle V as long as the near-infrared camera 41a can capture at least the face of a driver of the vehicle V sitting on the driver's seat Se. For example, the near-infrared camera 41a can be mounted to the steering column of the vehicle V or to the inner surface of the ceiling of the vehicle V or to the rearview mirror of the vehicle V.

The thermal image measurement device 42 is configured to periodically measure the temperature distribution of the body surface of a driver sitting on the driver's seat Se of the vehicle V, and periodically output the measured temperature distributions to the HCU 40. For example, the thermal image measurement device 42 includes a thermography camera that measures all radiation from the surface of the upper half body of the driver as an image showing the temperature distribution of the surface of the upper half body of the driver, and outputs the temperature-distribution image to the HCU 40.

Note that the camera unit 41 and the thermal image measurement device 42 can be configured to share a camera, so that the camera can be configured to periodically capture both the driver image and the temperature distribution image of the upper half body of the driver.

The operation device 43 is configured such that operations of the operation device 43 by a driver or at least one occupant enables various items of information to input to the HCU 40. For example, the operation device 43 is comprised of switches including steering switches mounted to the spokes of a steering wheel of the vehicle V, a touch screen layered on the top of a computer display provided in, for example, an instrument panel of the vehicle V or a navigation system of the vehicle V, and/or an audio input device provided in, for example, the navigation system.

The HCU 40 is constructed mainly by a computer including at least one processor 40a, such as a CPU, a memory unit 40b including at least one of non-transitory tangible storage media including a RAM and a ROM, an input/output (I/O) interface 40c, and other peripheral devices. The at least one processor 40a, memory unit 40b, and I/O interface 40c are communicably connected to each other via, for example a bus. The ROM is an example of a non-volatile memory.

At least part of all functions provided by the HCU 40 can be implemented by the at least one processor 40a; the at least one processor 40a, which will be simply referred to as the processor 40a, can be comprised of

(1) At least one programmable processing unit, i.e. at least one programmable logic circuit

(2) At least one hardwired logic circuit

(3) At least one hardwired-logic and programmable-logic hybrid circuit

For example, various programs for causing the processor 40a to perform the various functions, i.e. various routines, associated with reduction of driver's fatigue reduction and recovery from driver's fatigue are stored in the memory unit 40b. In addition, various data items usable by the processor 40a are also stored in the memory unit 40b. The processor 40a reads at least one of the various programs from the memory unit 40b, and executes the at least one program to thereby execute the function associated with reduction of driver's fatigue reduction and recovery from driver's fatigue, which corresponds to the at least one readout program.

That is, at least the HCU 40 can serve as a fatigue reduction facilitating apparatus according to the present disclosure. The combination of the HCU 40 and at least one of the components 5, 6, 41, 42, and 43 can also serve as the fatigue reduction facilitating apparatus according to the present disclosure.

The communication module 5 enables the HCU 40 and the mobile terminal 2 to radio communicate with each other in accordance with, for example, the near field communications standards. That is, the communication module 5 receives various information items and/or signals transmitted from the mobile terminal 2, and transmits the received information items and/or signals to the HCU 40. Similarly, the communication module 5 receives various information items and/or signals transmitted from the HCU 40, and transmits the received information items and/or signals to the mobile terminal 2.

The hot-cold stimulator 6 is installed in, for example, the driver's seat Se. The hot-cold stimulator 6 includes, for example, a hot-cold stimulation module 6a and an air duct Du installed in, for example, the driver's seat Se. The hot-cold stimulator 6 is configured such that the hot-cold stimulation module 6a supplies conditioned air, i.e. adjusted air, into the air duct Du, so that the supplied air is guided through the air duct Du and thereafter is blown to a driver sitting on the driver's seat Se. This enables hot stimuli and/or cold stimuli to be applied to the driver sitting on the driver's seat Se. This facilitates the blood flow of the driver, thus facilitating reduction in fatigue of the driver.

For example, as illustrated in FIG. 2, the driver's seat Se is comprised of a base Se1, a cushion seat member Se2, a back rest Se3, and a head rest Se4.

The base Se1, which has, for example, a rectangular-parallelepiped shape, is mounted on a floorboard of the vehicle V. The cushion seat member Se2, which has, for example, a rectangular- or square-parallelepiped shape, is mounted on a top surface of the base Se1. The cushion seat member Se2 is configured to comfortably support a driver of the vehicle V sitting thereon. The back rest Se3, which has, for example, a rectangular-parallelepiped shape, is continuously joined to a rear upper end of the cushion seat member Se2 via a first joint J1 to extent obliquely upward and backward. The back rest Se3 is configured to comfortably support the back of a driver of the vehicle V sitting on the cushion seat member Se2 of the driver's seat Se.

The head rest Se4, which has, for example, a rectangular-parallelepiped shape, is continuously joined via a second joint J2 to an upper end of the back rest Se3. The head rest Se4 is configured to comfortably support the head of a driver of the vehicle V sitting on the cushion seat member Se2 of the driver's seat Se.

For example, the hot-cold stimulation module 6a is installed in the base Se1 and the cushion seat member Se2, and the air duct Du is installed in the driver's seat Se so as to extend from, for example, the cushion seat member Se2 to the head rest Se4 through the back rest Se3.

For example, the air duct Du has an opening inlet communicably joined to the hot-cold stimulation module 6a. The air duct Du also includes branching ducts Du1, Du2, and Du3 branching off from predetermined respective portions of the air duct Du.

The branching duct Du1 is located in, for example, the first joint J1 to extend toward the waist of a driver sitting on the cushion seat member Se2. The branching duct Du1 has an extending opening end that is exposed from the first joint J1, so that the extending opening end of the branching duct Du1 serves as a waist vent WD that allows adjusted air supplied from the hot-cold stimulation module 6a to be blown toward the waist of the driver sitting on the cushion seat member Se2.

The branching duct Du2 is located in, for example, the back rest Se3 to extend toward the back of a driver sitting on the cushion seat member Se2. The branching duct Du2 has an extending opening end that is exposed from the back rest Se3, so that the extending opening end of the branching duct Du2 serves as a back vent BD that allows adjusted air supplied from the hot-cold stimulation module 6a to be blown toward the back of a driver sitting on the cushion seat member Se2.

The branching duct Du3 is located in, for example, the head rest Se4 to extend toward the shoulder of a driver sitting on the cushion seat member Se2. The branching duct Du3 has an extending opening end that is exposed from the head rest Se4, so that the extending opening end of the branching duct Du3 serves as a shoulder vent SD that allows adjusted air supplied from the hot-cold stimulation module 6a to be blown toward the shoulder of a driver sitting on the cushion seat member Se2.

The hot-cold stimulation module 6a receives, from the HCU 40, a hot-cold request instruction indicative of, for example, at least one of

(1) Selection information indicative of selection of at least one of the vents WD, BD, and SD

(2) Temperature information indicative of the temperature of air to be blown from the selected at least one of the vents WD, BD, and SD

(3) Continuous period information indicative of the period to continue the blowing of air from the selected at least one of the vents WD, BD, and SD

That is, the hot-cold stimulation module 6a is configured to

1. Adjust air to be blown from at least one of the vents WD, BD, and SD in accordance with the selection information, temperature information, and the continuous period information

2. Blow adjusted air from the selected at least one of the vents WD, BD, and SD, thus applying hot stimuli and/or cold stimuli to a driver of the vehicle V sitting on the driver's seat Se

Note that the fatigue reduction facilitating system 1 of the first embodiment is configured to apply hot stimuli and/or cold stimuli to a driver of the vehicle V sitting on the driver's seat Se using conditioned air blown from the hot-cold stimulator 6, but the present disclosure is not limited to this configuration. Specifically, the fatigue reduction facilitating system 1 can include a plurality of thermoelectric pads, such as Peltier-effect pads or heating-wire pads, placed on predetermined respective stimulus parts, such as waist, back, and shoulder, of a driver sitting on the driver's seat Se. The fatigue reduction facilitating system 1 controls how at least one pad selected from the thermoelectric pads is powered to thereby switch heating or cooling the corresponding at least one selected part, thus applying hot stimuli and/or cold stimuli to the at least one selected part of the driver.

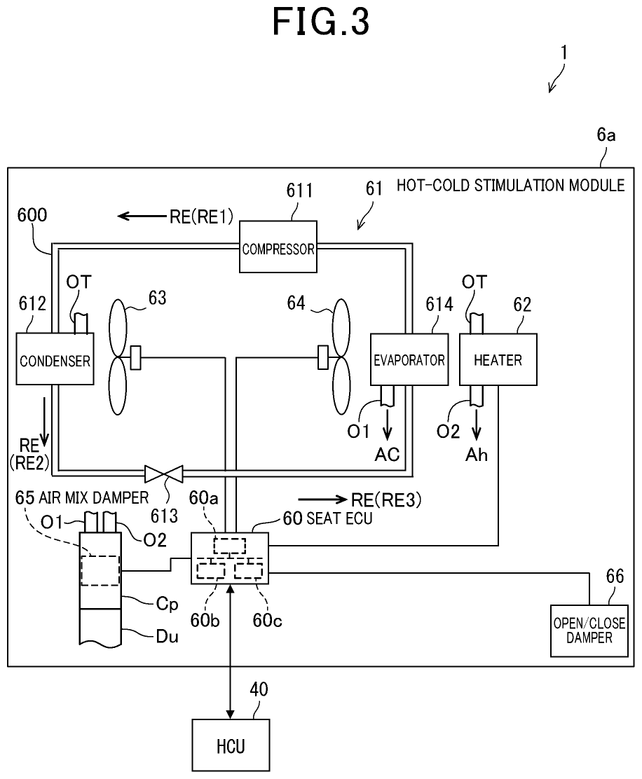

Next, the following describes an example of the schematic configuration of the hot-cold stimulation module 6a of the hot-cold stimulator 6 with reference to FIG. 3.

Referring to FIG. 3, the hot-cold stimulation module 6a includes, for example, a seat electronic control unit (ECU) 60, a refrigeration cycle system 61, a heating device 62, a first blower 63, a second blower 64, an air mix damper 65, open/close dampers 66, and a connection pipe CP. The connection pipe CP has opposing first and second opening ends, and the first opening end of the connection pipe CP is communicably joined to the opening inlet of the air duct Du.

The first blower 63 is configured to blow air to a condenser of the refrigeration cycle unit 61 described later, and the second blower 64 is configured to blow air to an evaporator 614 of the refrigeration cycle unit 61 described later.

The refrigeration cycle system 61 is configured to cool air using a vapor compression refrigeration cycle through which refrigerant is circulated. Specifically, the refrigeration cycle system 61 includes a closed conduit 600, a compressor 611, a condenser 612, a decompressor (thermal expansion valve) 613, and an evaporator 614. The closed conduit 600 is configured such that a refrigerant RE flows to be circulated therethrough. On the closed conduit 600, the compressor 611, condenser 612, decompressor 613, and evaporator 614 are mounted to be communicable through the closed conduit 600. The refrigeration cycle system 61 can use, as the refrigerant RE circulating through the closed conduit 600, chlorofluocarbonic gas with a low global warming potential, such as R134a refrigerant or R152a refrigerant, or use HC gas, such as propane gas.

The compressor 611 is an electric compressor driven based on a direct-current (DC) voltage supplied from a battery B installed in the vehicle V.

The compressor 611, which is activated based on the supplied DC voltage, is configured to compress, i.e. pressurize, the refrigerant RE supplied from the evaporator 614 described later through the closed conduit 600 thereinto to thereby increase the temperature and pressure of the refrigerant RE. Note that the refrigerant RE discharged from the compressor 611 will also be referred to as a refrigerant RE1. Then, the compressor 611 supplies the high-pressure and high-temperature refrigerant RE(RE1) to the condenser 612 via the closed conduit 600. Note that the hot-cold stimulation module 6a can use, as the compressor 611, a compressor installed in the vehicle V and driven by an engine of the vehicle V.

The condenser 612 serves as a heat exchanger or heat dissipator, and has an outlet pipe OT joined to the heating device 62. The condenser 612 is configured to exchange the refrigerator RE (RE1) supplied from the compressor 611 with the air supplied from the first blower 63 to thereby dissipate the heat of the refrigerant RE (RE1). This results in the temperature of the refrigerant RE (RE1) being reduced, so that the refrigerant RE (RE1) having a reduced temperature is supplied from the condenser 612 to the decompressor 613 as a refrigerant RE (RE2). In addition, the condenser 612 supplies the air supplied from the first blower 63, which has been heated by heat exchange with the refrigerant RE (RE1), to the heating device 62 via the outlet pipe OT.

The decompressor 613 is configured to depressurize the refrigerant RE (RE2) supplied from the condenser 612 through the closed conduit 600 thereinto to thereby decrease the temperature of the refrigerant RE (RE2) to a lower temperature, and also decrease the pressure of the refrigerant RE (RE2) to a lower pressure. Then, the decompressor 613 discharges the low-pressure and low-temperature refrigerant RE to the evaporator 614 via the closed conduit 600 as a refrigerant RE (RE3).

The evaporator 614 serves as an endothermic unit, and has an outlet O1 communicably coupled to the second opening end of the connection pipe CP. The evaporator 614 is configured to exchange the refrigerator RE (RE3) supplied from the decompressor 613 with the air supplied from the second blower 64 to thereby dissipate the heat of the air supplied from the second blower 64. This results in the temperature of the air being reduced. The refrigerator RE (RE3), whose temperature is increased based on the exchange operation by the evaporator 614, is supplied from the evaporator 614 into the compressor 611.

In addition, the evaporator 614 is configured to supply the air supplied from the second blower 64, which has been cooled by the heat exchange with the refrigerant RE (RE3), into the connection pipe CP via the second opening end of the connection pipe CP.

That is, the refrigeration cycle system 61 is configured to cool the air supplied from the second blower 64 while causing the refrigerant RE to circulate through the closed conduit 600, thus supplying the cooled air, which will be referred to as cooled air Ac, outputted from the evaporator 614 into the connection pipe CP via the second opening end of the connection pipe CP.

The heating device 62 is comprised of an electric heater, such as a positive temperature coefficient (PTC) heater or a hot-wire heater, and is configured to heat the air that has been heat exchange with the refrigerant in the condenser 612 to thereby heat the air, i.e. further increase the temperature of the air. Then, the heating device 62 supplies the heated air, which will be referred to as heated air Ah, into the connection pipe CP via the second opening end of the connection pipe CP.

The air mix damper 65 is disposed in the connection pipe CP that communicably couples the air duct Du to the outlet O1 of the evaporator 614 and the outlet O2 of the heating device 62 as set forth above. The air mix damper 65 is also controllably connected to the seat ECU 60.

The air mix damper 65 is comprised of, for example, an actuator and a door member, and is configured to adjust the amount of the cooled air Ac from the outlet O1 of the evaporator 614 to be supplied into the air duct Du and the amount of the heated air Ah from the outlet O2 of the heating device 62 to be supplied into the air duct Du under control of the seat ECU 60, thus adjusting, for example, the mixing ratio of the amount of the cooled air Ac and the amount of the heated air Ah, and the temperature of the mixture of the cooled air Ac and the heated air Ah. Then, the air mix damper 65 supplies the mixture of the cooled air Ac and the heated air Ah as conditioned air into the air duct Du. Adjustment of the temperature of the conditioned air to be supplied into the air duct Du enables the temperature of hot and cold stimuli applied to a driver of the vehicle V sitting on the driver's seat Se to be adjusted.

The open/close dampers 66 are disposed in the respective vents WD, BD, and SD of the air duct Du, and are controllably connected to the seat ECU 60. Each of the open/close dampers 66 is comprised of, for example, an actuator and a door member, such as a valve member, and is configured to open or close the corresponding one of the vents WD, BD, and SD.

This configuration of the open/close dampers 66 and the seat ECU 60 is capable of

(1) Selecting at least one the vents WD, BD, and SD for blowing the conditioned air to the corresponding part of a driver sitting on the driver's seat Se

(2) Adjusting a continuous period to continue the blowing of the conditioned air from at least one of the vents WD, BD, and SD to the corresponding part of a driver sitting on the driver's seat Se

Selecting at least one of the vents WD, BD, and SD for blowing the conditioned air enables the vents WD, BD, and SD for blowing the conditioned air to be changed, making it possible to change the parts of a driver sitting on the driver's seat Se to which hot stimuli and/or cold stimuli are applied.

As described above, the stimulus parts of a driver sitting on the driver's seat Se to which hot stimuli and/or cold stimuli are to be applied according to the first embodiment are determined as the waist, back, and shoulder of the driver. That is, the conditioned air blown from the vent WD applies hot stimuli or cold stimuli to the waist of the driver, the conditioned air blown from the vent BD applies hot stimuli or cold stimuli to the back of the driver, and the conditioned air blown from the vent SD applies hot stimuli or cold stimuli to the shoulder of the driver.

Adjusting the continuous period to continue the blowing of the conditioned air from at least one of the vents WD, BD, and SD to the corresponding part of a driver sitting on the driver's seat Se enables adjustment of the period of the corresponding part of the driver being hot stimulated or cold stimulated.

The seat ECU 60 is constructed mainly by a computer including at least one processor 60a, such as a CPU, a memory 60b including at least one of non-transitory tangible storage media including a RAM and a ROM, an input/output (I/O) interface 60c, and other peripheral devices. The at least one processor 60a, memory 60b, and I/O interface 60c are communicably connected to each other via, for example a bus.

At least part of all functions provided by the seat ECU 60 can be implemented by the at least one processor 60a; the at least one processor 60a, which will be simply referred to as the processor 60a, can be comprised of

(1) The combination of at least one programmable processing unit, i.e. at least one programmable logic circuit

(2) At least one hardwired logic circuit

(3) At least one hardwired-logic and programmable-logic hybrid circuit

The seat ECU 60 is communicably connected to the HCU 40.

For example, various programs for causing the processor 60a to perform the various functions, i.e. various routines, associated with hot-cold stimuli are stored in the memory 60b. In addition, various data items usable by the processor 60a are also stored in the memory 60b. The processor 60a reads at least one of the various programs from the memory 60b, and executes the at least one program to thereby execute the function associated with hot-cold stimuli, which corresponds to the at least one readout program.

In particular, the seat ECU 60 is configured to receive a hot-cold request instruction from the HCU 40, and adjust at least one of the temperature of the conditioned air, the continuous period of the conditioned air to be applied to a driver sitting on the driver's seat Se, and at least one part of the driver to which hot stimuli or cold stimuli are applied.

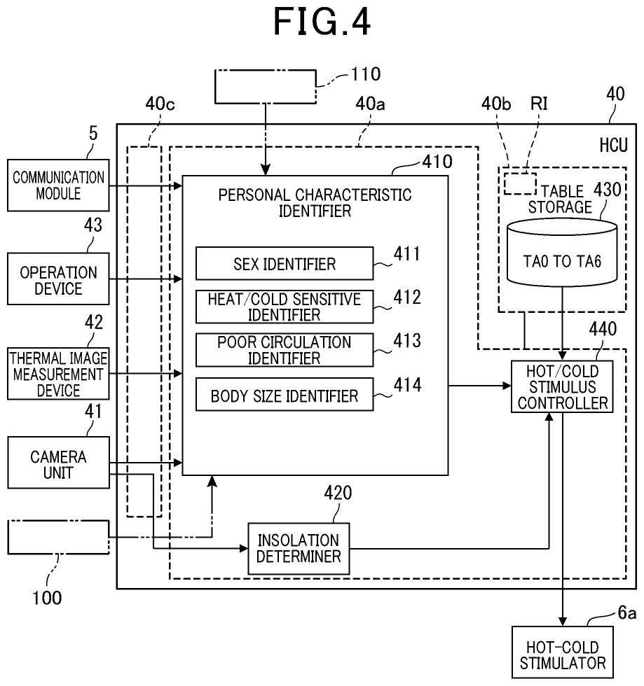

Next, the following describes an example of the functional configuration of the HCU 40 with reference to FIG. 4.

The HCU 40 functionally includes a personal characteristic identifier 410, an insolation determiner 420, a table storage 430, and a hot/cold stimulus controller 440. The personal characteristic identifier 410 includes, for example, a sex identifier 411, a heat/cold sensitive identifier 412, a poor-circulation identifier 413, and a body size identifier 414.

The processor 40a of the HCU 40 can be configured such that at least one programmable logic circuit implements at least one of the functions 410 to 440, at least one hardwired-logic circuit implements at least one of the functions 410 to 440, or at least one hardwired-logic and programmable-logic hybrid circuit implements at least one of the functions 410 to 440.

The personal characteristic identifier 410 is configured to identify the personal characteristics of a driver sitting on the driver's seat Se. For example, the personal characteristics identified by the personal characteristic identifier 410 include

1. The sex (gender) of a driver sitting on the driver's seat Se as the first personal characteristic item

2. The information on whether the driver is sensitive to heat or cold, i.e. the driver is hot-natured or cold-natured, or is less-sensitive to any of heat and cold as the second personal characteristic item

3. The information on whether the driver has poor circulation as the third personal characteristic item

4. The height of the driver as the fourth personal characteristic item

5. The weight of the driver as the fifth personal characteristic item

The height and weight of the driver collectively show the body size of the driver.

The sex identifier 411 identifies the sex of a driver sitting on the driver's seat Se, the heat/cold sensitive identifier 412 identifies the information on the driver being sensitive to heat or cold, the poor-circulation identifier 413 identifies the information on the driver having poor circulation, and the body size identifier 414 identifies the body size of the driver.

For example, the memory unit 40b stores feature quantity templates, i.e. feature quantity patterns, of various persons that can drive vehicles. Then, the sex identifier 411 can be configured to perform pattern matching of one or more driver images captured by the camera unit 41 with each of the feature patterns stored in the memory unit 40b to thereby identify the sex of a driver sitting on the driver's seat Se.

The sex identifier 411 can be configured to send request information for prompting input of the sex of the driver to the mobile terminal 2 or the operation device 43, and cause the mobile terminal 2 or the operation device 43 to visibly or audibly output the request information, thus prompting input of the driver's sex, i.e. male or female, for the driver. In this example, when sex information about the driver's sex is entered by the driver in the mobile terminal 2 or the operation device 43, the entered sex information is sent from the mobile terminal 2 or the operation device 43 to the sex identifier 411 of the HCU 40. Then, the sex identifier 411 receives the entered sex information via the communication module 5, and identifies the sex of the driver based on the entered sex information.

The heat/cold sensitive identifier 412 can be configured to send, to the mobile terminal 2 or the operation device 43, request information for prompting input of hot/cold-natured information about whether the driver is sensitive to heat or cold or less-sensitive to any of heat and cold, and cause the mobile terminal 2 or the operation device 43 to visibly or audibly output the request information, thus prompting, for the driver, input of the hot/cold-natured information about whether the driver is sensitive to heat or cold or less-sensitive to any of heat and cold.

In this example, when the hot- or cold-natured information is entered by the driver in the mobile terminal 2 or the operation device 43, the entered hot- or cold-natured information is sent from the mobile terminal 2 or the operation device 43 to the heat/cold sensitive identifier 412 of the HCU 40. Then, the heat/cold sensitive identifier 412 receives the entered hot- or cold-natured information via the communication module 5, and identifies whether the driver is sensitive to heat or cold or less-sensitive to any of heat and cold.

The heat/cold sensitive identifier 412 can be configured to communicate with an air-conditioner installed in the vehicle V to thereby receive a current value of the setting temperature of the air-conditioner and/or an average value of the setting temperature, which is set by driver's operation of an operation panel of the air-conditioner.

Note that the heat/cold sensitive identifier 412 can be configured to communicate with the mobile terminal 2 if the setting temperature of the air-conditioner can be remotely controlled by the mobile terminal 2 and a current value of the setting temperature of the air-conditioner is set based on driver's operation of the mobile terminal 2. Then, the heat/cold sensitive identifier 412 can be configured to receive a current value of the setting temperature of the air-conditioner and/or an average value of the setting temperature of the air-conditioner from the mobile terminal 2.

In response to receiving the current value and/or the average value of the setting temperature of the air-conditioner, the heat/cold sensitive identifier 412 can be configured to compare the current value or average value of the setting temperature with a corresponding reference temperature threshold to thereby determine whether the current value or average value of the setting temperature is higher or lower by at least predetermined levels than the reference temperature threshold.

Upon determining that the current value or average value of the setting temperature is higher or lower by the at least predetermined levels than the reference temperature threshold, the heat/cold sensitive identifier 412 can be configured to identify that the driver is sensitive to heat or cold based on a result of the determination of whether the current value or average value of the setting temperature is higher or lower by the at least predetermined levels than the reference temperature threshold.

As another example, the heat/cold sensitive identifier 412 can be configured to identify the driver being sensitive to heat based on, for example, determination that at least one of a value of the height and a value of the weight of a driver sitting on the driver's seat Se, which are inputted from the mobile terminal 2 or the operation device 43, is equal to or more than a predetermined threshold value.

As a further example, the heat/cold sensitive identifier 412 can be configured to identify the driver being sensitive to heat based on, for example, determination that a value of a physical body parameter of the driver is equal to or more than a predetermined threshold value; the value of the physical body parameter is determined based on a value of the height and a value of the weight of the driver inputted from the mobile terminal 2 or the operation device 43.

As a still further example, the heat/cold sensitive identifier 412 can be configured to identify whether the driver is sensitive to heat or cold or less-sensitive to any of heat and cold based on, for example, information inputted from the mobile terminal 2 or the operation device 43.

The poor-circulation identifier 413 can be configured to receive the temperature distributions of the body surface of a driver sitting on the driver's seat Se, and identify whether the driver has poor circulation based on the received temperature distributions of the body surface of the driver. For example, the poor-circulation identifier 413 can be configured to obtain, from the received temperature distributions of the body surface of the driver, the temperature of at least one terminal part, such as a finger or a hand, of the driver. Then, the poor-circulation identifier 413 can be configured to determine whether the temperature of the at least one terminal part of the driver is equal to or less than a predetermined threshold temperature, and identify that the driver has poor circulation upon determining that the temperature of the at least one terminal part of the driver is equal to or less than the predetermined threshold temperature.

As another example, the poor-circulation identifier 413 can be configured to send request information for prompting input of information on whether the driver has poor circulation, and cause the mobile terminal 2 or the operation device 43 to visibly or audibly output the request information, thus prompting, for the driver, input of the information on whether the driver has poor circulation.

In this example, when poor-circulation information indicative of whether the driver has poor circulation is entered by the driver in the mobile terminal 2 or the operation device 43, the entered poor-circulation information is sent from the mobile terminal 2 or the operation device 43 to the poor-circulation identifier 413 of the HCU 40. Then, the poor-circulation identifier 413 receives the entered poor-circulation information via the communication module 5, and identifies whether the driver has poor circulation.

The body size identifier 414 can be configured to send request information for prompting input of a value of each of the height and weight of the driver, and cause the mobile terminal 2 or the operation device 43 to visibly or audibly output the request information, thus prompting, for the driver, input of a value of each of the height and weight of the driver.

In this example, when the value of each of the height and weight of the driver is entered by the driver in the mobile terminal 2 or the operation device 43, the entered value of each of the height and weight of the driver is sent from the mobile terminal 2 or the operation device 43 to the body size identifier 414 of the HCU 40. Then, the body size identifier 414 receives the entered value of each of the height and weight of the driver via the communication module 2 or the operation device 43 to thereby identify the body size of the driver.

As another example, the body size identifier 414 can be configured to extract, from one or more driver images captured by the camera unit 41, the position of the face or head of the driver, and estimate, i.e. identify, the height of the driver based on the position of the face or head of the driver. Additionally, a pressure sensor 100 can be provided in the driver's seat Se of the vehicle V for measuring the pressure applied to the cushion seat member Se2 when the driver is sitting on the driver's seat Se. At that time, the body size identifier 414 can be configured to receive, from the pressure sensor 100, the measured pressure, and estimate, i.e. identify, the weight of the driver based on the measured pressure.

Note that, as described above, the first to fifth personal characteristic items identified by the identifiers 411 to 414 show the personal characteristics, which will also be referred to as personal characteristic information, of the driver sitting on the driver's seat Se.

Usually, the vehicle V includes a keyless ignition system 110 that enables a driver of the vehicle V to power up the vehicle V by pushing a button if identification data (ID) of an electronic key detected within the vehicle V is matched with a previously registered ID in the system 110 so that the ID of the electronic key is verified.

At that time, the personal characteristic identifier 410 of the HMI system 4 can be configured to store the personal characteristic information of the driver in the non-volatile memory, such as the ROM, of the memory unit 40b such that the personal characteristic information of the driver correlates with the ID of the electronic key of the driver.

This configuration enables, each time the same driver with the electronic key sits the driver's seat Se of the vehicle V, the personal characteristic information about the driver stored to correlate with the ID of the electronic key in the memory 40b to be easily red from the memory 40b without driver's input of some items of the personal characteristic information.

As described above, the personal characteristic identifier 410 of the first embodiment is configured to use the communication module 5, the mobile terminal 2, the camera unit 41, the thermal image measurement device 42, and the operation device 43 to thereby identify the personal characteristic information, but the present disclosure is not limited to this configuration. Specifically, the personal characteristic identifier 410 can be configured not to use at least one of the communication module 5, the mobile terminal 2, the camera unit 41, the thermal image measurement device 42, and the operation device 43 if it is unnecessary to identify at least one of the first to fifth personal characteristic items of the personal characteristic information.

Additionally, the insolation determiner 420 is configured to determine whether solar radiation is hitting a driver sitting on the driver's seat Se.

For example, the insolation determiner 420 can be configured to perform pattern matching of a current driver image captured by the camera unit 41 with each of the feature patterns stored in the memory unit 40b to thereby identify the region of a predetermined part, such as the face, of a driver sitting on the driver's seat Se. Then, the insolation determiner 420 can be configured to determine whether the intensity of each pixel in the identified region in the driver image is equal to or higher than a predetermined light intensity, and determine that solar radiation is hitting the predetermined part of the driver upon determining that the intensity of each pixel in the identified region in the driver image is equal to or higher than the predetermined light intensity.

As described above, the seat ECU 60 is configured to control, based on the hot-cold request instruction sent from the HCU 40, the air mix damper 65 and each of the open/close dampers 66 of the respective vents WD, BD, and SD to thereby perform

(1) An application of a hot stimulus based on the conditioned air with the controlled high temperature to at least one of the stimulus parts of waist, back, and shoulder of a driver sitting of the driver's seat Se via the corresponding at least one of the vents WD, BD, and SD

(2) An application of a cold stimulus based on the conditioned air with the controlled low temperature to at least one of the stimulus parts of waist, back, and shoulder of a driver sitting of the driver's seat Se via the corresponding at least one of the vents WD, BD, and SD

The hot-cold request instruction can include, for example, a default stimulus mode based on default hot-cold conditions.

The default stimulus mode is determined to cause the seat ECU 60 to

(1) Apply one cycle of alternate hot and cold stimuli to all the stimulus parts of waist, back, and shoulder in synchronization with each other

(2) Set the temperature of each hot stimulus to a predetermined first common setting temperature TE1 for all the stimulus parts of waist, back, and shoulder

(3) Set the application period of each hot stimulus to a predetermined common first setting period P1 for all the stimulus parts of waist, back, and shoulder

(4) Set the temperature of each cold stimulus to a predetermined second common setting temperature TE2 for all the stimulus parts of waist, back, and shoulder

(5) Set the application period of each cold stimulus to a predetermined common second setting period P2 for all the stimulus parts of waist, back, and shoulder

(6) Repeat the cycle of the alternate hot and cold stimuli a predetermined number of times

That is, the default stimulus part(s) to which alternate hot and cold stimuli are applied in the default stimulus mode are set to all the stimulus parts of waist, back, and shoulder.

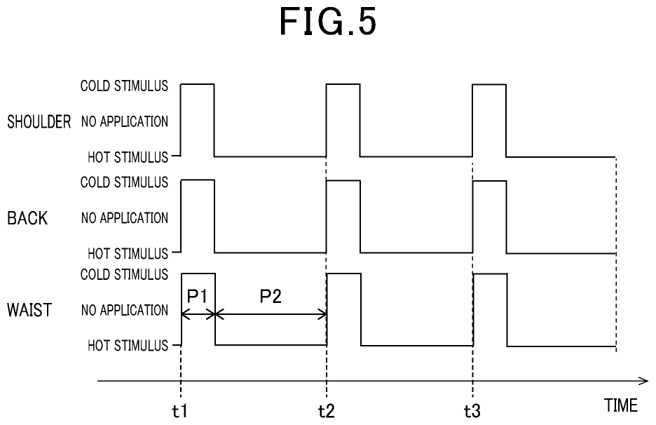

FIG. 5 schematically illustrates how the hot-cold stimulator 6 sequentially performs the cycle of the alternate hot and cold stimuli. In FIG. 5, the horizontal axis represents time, and the vertical axis represents an application of a hot stimulus, an application of a cold stimulus, and no application of hot and cold stimuli.

For example, as illustrated in FIG. 5, the seat ECU 60 controls, based on the default stimulus mode of the hot-cold request instruction, the air mix damper 65 and each of the open/close dampers 66 of the respective vents WD, BD, and SD to thereby perform

(1) A first cycle of an application of a hot stimulus at the first common setting temperature TE1 to each of the waist, back, and shoulder of the driver for the first common setting period P1 at time t1, and, thereafter, an application of a cold stimulus at the second common setting temperature TE2 to each of the waist, back, and shoulder of the driver for the second common setting period P2

(2) A second cycle of an application of a hot stimulus at the first common setting temperature TE1 to each of the waist, back, and shoulder of the driver for the first common setting period P1 at time t2, and, thereafter, an application of a cold stimulus at the second common setting temperature TE2 to each of the waist, back, and shoulder of the driver for the second common setting period P2

Although above description shows that the hot-cold stimulator 6 performs the cycle of alternate hot and cold stimuli only two times, but the hot-cold stimulator 6 can perform the cycle of alternate hot and cold stimuli the predetermined number of times.

The table storage 430 can be provided in, for example, the non-volatile memory, such as the ROM, of the memory unit 40b, and include a plurality of tables, for example, tables TA0, TA1, TA2, TA3, TA4, and TA5; the table TA0 represents the above default setting values of the default stimulus mode, and the tables TA1 to TA5 represents adjusted information with respect to the default setting values of the default stimulus mode prepared for the respective first to fifth personal characteristic items.

Each of the tables TA1 to TA5 prepared for the respective first to fifth personal characteristic items schematically stores

(1) An adjusted value of temperature for an application of each hot stimulus relative to a corresponding setting temperature

(2) An adjusted value of application period of each hot stimulus relative to a corresponding default application time

(3) At least one adjusted part to which each hot stimulus is to be applied with respect to the default parts, i.e. all the parts of the waist, back, and shoulder of the driver

(4) An adjusted value of temperature for an application of each cold stimulus relative to a corresponding setting temperature

(5) An adjusted value of application period of each cold stimulus relative to a corresponding default application time

(6) At least one adjusted part to which each cold stimulus is to be applied with respect to the default parts, i.e. all the parts of the waist, back, and shoulder of the driver

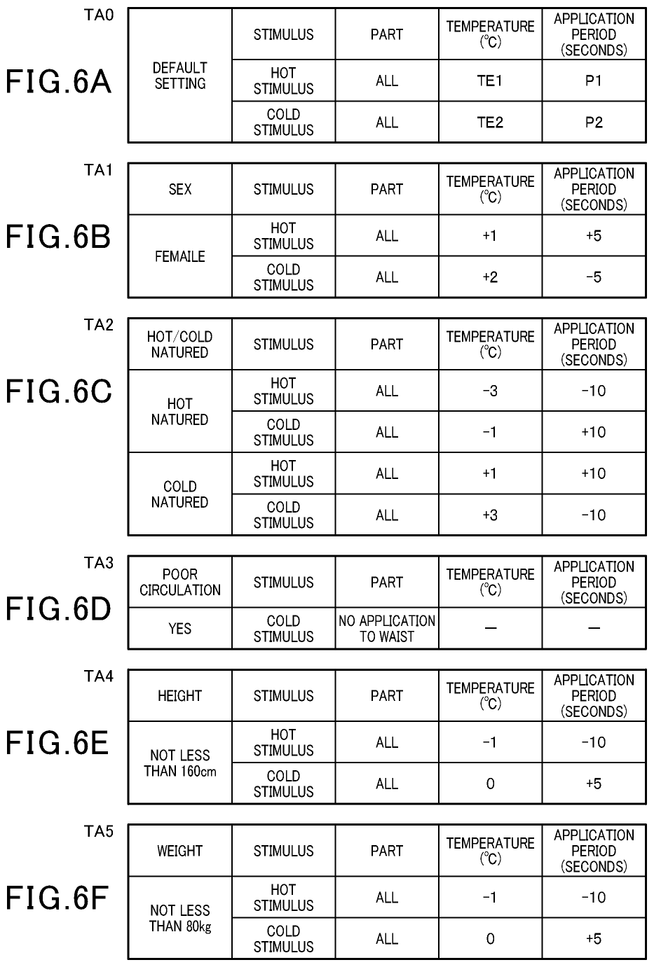

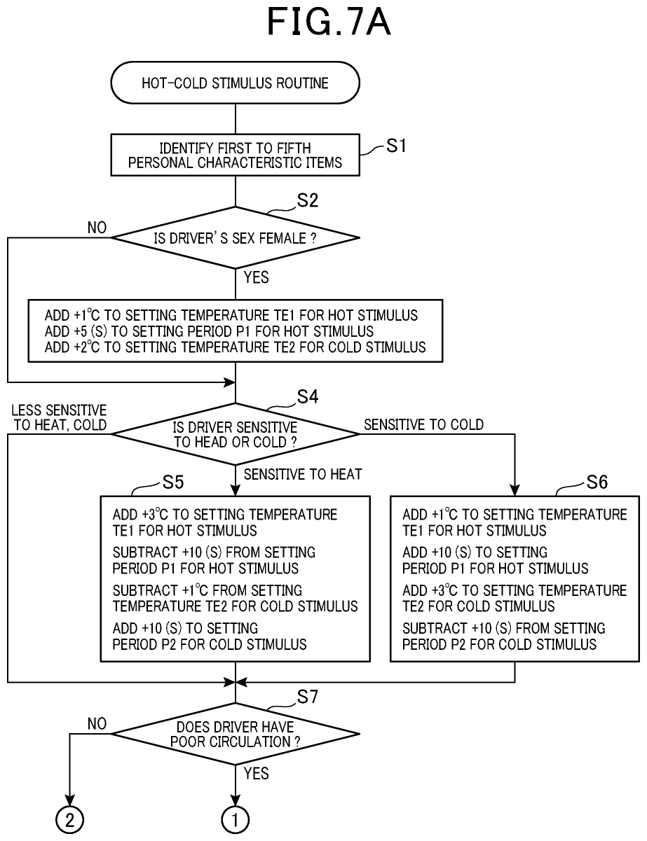

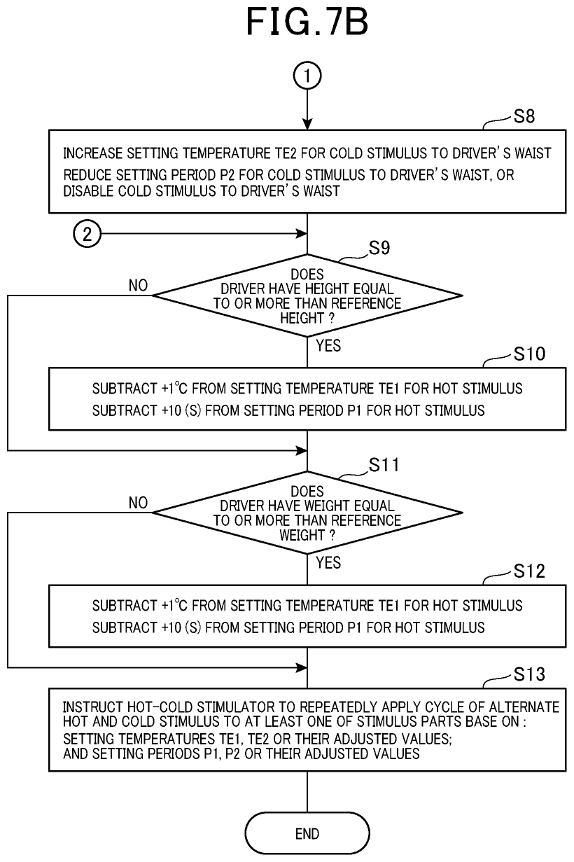

The following describes, in more details, how each of the tables TA0 to TA5 store information with reference to FIGS. 6A to 6F. In each of FIGS. 6A to 6F, the unit of temperature is degrees (° C.), and the unit of application time is seconds (S).

As illustrated in FIG. 6A, the table TA0 for the default stimulus mode includes

(1) The first common setting temperature TE1 (° C.) for an application of each hot stimulus

(2) The second common setting temperature TE2 (° C.) for an application of each cold stimulus

(3) The first common setting period P1 in seconds for an application of each hot stimulus

(4) The second common setting period P2 in seconds for an application of each cold stimulus

Note that each of the first common setting temperature TE1, second common setting temperature TE2, first common setting period P1, and second common setting period P2 are determined such that they respectively seem to facilitate the blood flow of a driver sitting on the driver's seat Se, thus facilitating reduction in fatigue of the driver.

For example, the fatigue reduction facilitating system 1 applied hot and cold stimuli to many tested drivers while changing the temperature and period of each hot stimulus and changing the temperature and period of each cold stimulus. Then, the fatigue reduction facilitating system 1 sampled, from the many tested drivers,

(1) Effective temperatures in all the temperatures of the hot stimuli in reduction of tested-driver's fatigue

(2) Effective application periods in all the application periods of the hot stimuli in reduction of tested-driver's fatigue

(3) Effective temperatures in all the temperatures of the cold stimuli in reduction of tested-driver's fatigue

(4) Effective application periods in all the application periods of the cold stimuli in reduction of tested-driver's fatigue

After the sampling, the fatigue reduction facilitating system 1 has stored, in the table T0,

(1) An average value or a mode value from the sampled effective temperatures of the corresponding hot stimuli as the first common setting temperature TE1

(2) An average value or a mode value from the sampled effective application periods of the corresponding hot stimuli as the first common setting period P1

(3) An average value or a mode value from the sampled effective temperatures of the corresponding hot stimuli as the first common setting temperature TE1

(4) An average value or a mode value from the sampled effective application periods of the corresponding cold stimuli as the second common setting period P2

For example, the first common setting temperature TE1 is set to 40° C., and the second common setting temperature TE2 is set to 15° C. The first common setting period P1 is set to, for example, 300 seconds, and the second common setting period P2 is set to, for example, 40 seconds.

As described above, the default hot-cold conditions in the default stimulus mode include

(1) The stimulus part(s) to which hot and cold stimuli are applied is set to all the stimulus parts

(2) The temperature of each hot stimulus is set to the first common setting temperature TE1

(3) The temperature of each cold stimulus is set to the second common setting temperature TE2

(4) The application period for which each hot stimulus is applied is set to the first common setting period P1

(5) The application period for which each cold stimulus is applied is set to the second common setting period P2

As illustrated in FIG. 6B, the table TA1 for the first personal characteristic item, i.e. the sex, being identified as female includes

(1) The adjusted value of +1° C., i.e. an increment of 1° C., of temperature for an application of each hot stimulus relative to the first common setting temperature TE1

(2) The adjusted value of +5 seconds, i.e. an increment of 5 seconds, of application period of each hot stimulus relative to the first common setting period P1

(3) No adjustment of the default parts, i.e. all the parts of the waist, back, and shoulder of the driver, to which the hot stimuli are to be applied

(4) The adjusted value of +2° C., i.e. an increment of 2° C. of temperature for an application of each cold stimulus relative to the second common setting temperature TE2

(5) The adjusted value of −5 seconds, i.e. a decrement of 5 seconds, of application time of each cold stimulus relative to the second common setting period P2

(6) No adjustment of the default parts, i.e. all the parts of the waist, back, and shoulder of the driver, to which the cold stimuli are to be applied

Note that FIG. 6B eliminates illustration of a table for the first personal characteristic item, i.e. the sex, being identified as male. The reasons are as follows.

Specifically, an application of each hot stimulus for the driver being identified as mal is carried out for each of the default stimulus parts at the first common setting temperature TE1 for the first common setting period P1. Similarly, an application of each cold stimulus for the driver being identified as male is carried out for each of the default stimulus parts at the second common setting temperature TE2 for the second common setting period P2.

As illustrated in FIG. 6C, the table TA2 for driver being identified to be sensitive to heat as the second personal characteristic item includes

(1) The adjusted value of −3° C., i.e. a decrement of 3° C., of temperature for an application of each hot stimulus relative to the first common setting temperature TE1

(2) The adjusted value of −10 seconds, i.e. a decrement of 10 seconds, of application period of each hot stimulus relative to the first common setting period P1

(3) No adjustment of the default parts, i.e. all the parts of the waist, back, and shoulder of the driver, to which the hot stimuli are to be applied

(4) The adjusted value of −1° C., i.e. a decrement of 1° C. of temperature for an application of each cold stimulus relative to the second common setting temperature TE2

(5) The adjusted value of +10 seconds, i.e. an increment of 10 seconds, of application period of each cold stimulus relative to the second common setting period P2

(6) No adjustment of the default parts, i.e. all the parts of the waist, back, and shoulder of the driver, to which the cold stimuli are to be applied

Additionally, the table T2 for driver being identified to be sensitive to cold as the second personal characteristic item includes

(1) The adjusted value of +1° C., i.e. an increment of 1° C., of temperature for an application of each hot stimulus relative to the first common setting temperature TE1

(2) The adjusted value of +10 seconds, i.e. an increment of 10 seconds, of application period of each hot stimulus relative to the first common setting period P1

(3) No adjustment of the default parts, i.e. all the parts of the waist, back, and shoulder of the driver, to which the hot stimuli are to be applied

(4) The adjusted value of +3° C., i.e. an increment of 3° C. of temperature for an application of each cold stimulus relative to the second common setting temperature TE2

(5) The adjusted value of −10 seconds, i.e. a decrement of 10 seconds, of application period of each cold stimulus relative to the second common setting period P2

(6) No adjustment of the default parts, i.e. all the parts of the waist, back, and shoulder of the driver, to which the cold stimuli are to be applied

As illustrated in FIG. 6D, the table TA3 for driver being identified to have poor circulation as the third personal characteristic item includes that an application of cold stimuli to the stimulus part of waist of the driver is disabled.

Other setting values for the driver having poor circulation can be used for the default values. Specifically, an application of hot stimuli for the driver being identified to have poor circulation is carried out for each of the default stimulus parts at the first common setting temperature TE1 for the first common setting period P1. Similarly, an application of cold stimuli for the driver being identified to have poor circulation is carried out for each of the adjusted stimulus parts of back and shoulder at the second common setting temperature TE2 for the second common setting period P2.

Note that the table TA3 for the driver being identified to have poor circulation is determined such that an application of cold stimuli to the stimulus part of waist of the driver is disabled, but the present disclosure is not limited thereto. Specifically, the table TA3 for the driver being identified to have poor circulation can be determined such that an application of weaker cold stimuli to the stimulus part of waist of the driver are carried out. For example, the table TA3 can include a positive adjusted value of temperature for an application of each cold stimulus relative to the second common setting temperature TE2, or a negative adjusted value of application period of each cold stimulus relative to the second common setting period P2.

As illustrated in FIG. 6E, the table TA4 for driver being identified to have a height equal to or more than a predetermined reference height, i.e. 160 centimetres, as the fourth personal characteristic item includes

(1) The adjusted value of −1° C., i.e. a decrement of 1° C., of temperature for an application of each hot stimulus relative to the first common setting temperature TE1

(2) The adjusted value of −10 seconds, i.e. a decrement of 10 seconds, of application period of each hot stimulus relative to the first common setting period P1

(3) No adjustment of the default parts, i.e. all the parts of the waist, back, and shoulder of the driver, to which the hot stimuli are to be applied

(4) No adjustment of temperature for an application of each cold stimulus relative to the second common setting temperature TE2

(5) The adjusted value of +5 seconds, i.e. an increment of 5 seconds, of application time of each cold stimulus relative to the second common setting period P2

(6) No adjustment of the default parts, i.e. all the parts of the waist, back, and shoulder of the driver, to which the cold stimuli are to be applied

As illustrated in FIG. 6F, the table TA5 for driver being identified to have a weight equal to or more than a predetermined reference weight, i.e. 80 kilograms, as the fifth personal characteristic item includes

(1) The adjusted value of −1° C., i.e. a decrement of 1° C., of temperature for an application of each hot stimulus relative to the first common setting temperature TE1

(2) The adjusted value of −10 seconds, i.e. a decrement of 10 seconds, of application period of each hot stimulus relative to the first common setting period P1

(3) No adjustment of the default parts, i.e. all the parts of the waist, back, and shoulder of the driver, to which the hot stimuli are to be applied

(4) No adjustment of temperature for an application of each cold stimulus relative to the second common setting temperature TE2

(5) The adjusted value of +5 seconds, i.e. an increment of 5 seconds, of application time of each cold stimulus relative to the second common setting period P2

(6) No adjustment of the default parts, i.e. all the parts of the waist, back, and shoulder of the driver, to which the cold stimuli are to be applied

The hot-cold stimulus controller 440 causes the seat ECU 60 of the hot-cold stimulator 6 to control the air mix damper 65 and each of the open/close dampers 66 of the respective vents WD, BD, and SD to thereby perform

(1) An application of each hot stimulus based on the conditioned air with the controlled high temperature to at least one of the stimulus parts of waist, back, and shoulder of a driver sitting of the driver's seat Se via the corresponding at least one of the vents WD, BD, and SD

(2) An application of each cold stimulus based on the conditioned air with the controlled low temperature to at least one of the stimulus parts of waist, back, and shoulder of a driver sitting of the driver's seat Se via the corresponding at least one of the vents WD, BD, and SD

That is, the hot-cold stimulus controller 440 sends the hot-cold request instruction to the seat ECU 60 of the hot-cold stimulator 6 to thereby control operations of the seat ECU 60, i.e. operations of the hot-cold stimulator 6.

Hereinafter, a routine, carried out by the hot-cold stimulus controller 440, of instructing the hot-cold stimulator 6 to perform an application of each hot stimulus and/or an application of each cold stimulus to a driver sitting on the driver's seat Se in order to reduce fatigue of the driver will also be referred to as a hot-cold stimulus routine.

For example, the hot-cold stimulus controller 440 can be configured to start the hot-cold stimulus routine in response to when the vehicle V is powered on based on the battery so that the HCU 40 is switched on. The hot-cold stimulus controller 440 can also be configured to estimate, based on the driver images captured by the camera unit 41, the level of fatigue of the driver, and start the hot-cold stimulus routine upon determination that the estimated level of fatigue of the driver becomes equal to or higher than a predetermined threshold level.

In addition, the hot-cold stimulus controller 440 can also be configured to start the hot-cold stimulus routine upon determination that a continuous travelling time of the vehicle V has reached a predetermined threshold time or a continuous travelling distance of the vehicle V has reached a predetermined threshold distance.

The hot-cold stimulus controller 440 can be configured to terminate the hot-cold stimulus routine in response to when the vehicle V is powered off so that the HCU 40 is switched off. The hot-cold stimulus controller 440 can also be configured to terminate the hot-cold stimulus routine upon determination that the estimated level of fatigue of the driver becomes lower than the predetermined threshold level.

In particular, the hot-cold stimulus controller 440 changes at least one of the default hot-cold conditions included in the hot-cold request instruction in accordance with the personal characteristics of the driver identified by the personal characteristic identifier 410.

In particular, as described above, the hot-cold stimulus controller 440 is capable of changing each of the default setting values, i.e. reference values, TE1, TE2, P1, and P2 of the respective temperature/application-period conditions included in the default hot-cold conditions based on a corresponding one of the adjusted values; the adjusted values are determined for the personal characteristics of the driver identified by the personal characteristic identifier 410. Note that the temperature/application-period conditions included in the default hot-cold conditions will also be respectively referred to as quantitative conditions.

Specifically, the hot-cold stimulus controller 440 changes at least one of the default hot-cold conditions included in the hot-cold request instruction in accordance with the tables TA1 to TA5 provided for the personal characteristics of the driver identified by the personal characteristic identifier 410.

The following describes how the hot-cold stimulus controller 440 changes at least one of the default hot-cold conditions included in the hot-cold request instruction.

As described above, the personal characteristic identifier 410 can identify the first to fifth personal characteristic items of the driver. When the personal characteristic identifier 410 identifies at least two items in the first to fifth personal characteristic items of the driver, the hot-cold stimulus controller 440 is configured to

(1) Extract at least one default setting value in the default setting values TE1, TE2, P1, and P2 from the table TA0

(2) Extract an adjusted value from each of the tables that respectively correspond to the two items

(3) Add the adjusted value for each of the two items to the corresponding one of the default setting values or subtract the adjusted value for each of the two items from the corresponding one of the default setting values, thus changing the corresponding one of the quantitative conditions included in the hot-cold request instruction

If the first to fifth personal characteristic items of the driver are illustrated by a single graph, it may be difficult for the hot-cold stimulus controller 440 to change the quantitative conditions.

In contrast, the hot-cold stimulus controller 440 of the first embodiment is configured to simply add the adjusted value for each of the two items to the corresponding one of the default setting values or subtract the adjusted value for each of the two items from the corresponding one of the default setting values, thus changing the corresponding one of the quantitative default hot-cold conditions included in the hot-cold request instruction. This configuration therefore simplifies adjustment of each of the default setting values.

The HCU 40 can include range information RI stored in the memory 40b; the range information RI includes a predetermined changeable range from a lower limit to an upper limit inclusive for each of the quantitative conditions; the changeable range for each of the quantitative conditions is set beforehand to be narrower than a range for the corresponding one of the quantitative conditions settable by the hot-cold stimulator 6.

That is, the hot-cold stimulus controller 440 preferably changes a value of each of the quantitative conditions within the corresponding changeable range in accordance with the range information RI.

For example, if the sum of the adjusted value and the default setting value for each of the quantitative conditions exceeds the upper limit of the corresponding changeable range, the hot-cold stimulus controller 440 preferably changes the default setting value to the upper limit of the corresponding changeable range. If the subtraction of the adjusted value from the default setting value for each of the quantitative conditions becomes lower than the lower limit of the corresponding changeable range, the hot-cold stimulus controller 440 preferably changes the default setting value to the lower limit of the corresponding changeable range.

This prevents the temperature of the conditioned air blown from the air duct Du from excessively increasing or from excessively decreasing, thus preventing the driver of the vehicle V from having an uncomfortable feeling due to the excessive increase or decrease in the temperature of the conditioned air independently of the personal characteristics of the driver. The upper and lower limits of each of the quantitative conditions can be determined such that they are located out of a predetermined driver-uncomfortable range where a value of the corresponding one of the quantitative conditions located within driver-uncomfortable range may cause the driver to have an uncomfortable feeling

For example, the changeable range from the lower limit to the upper limit inclusive for the temperature condition of each hot stimulus can be set to a range from 37° C. to 43° C. inclusive, and the changeable range from the lower limit to the upper limit inclusive for the application-period condition of each hot stimulus can be set to a range from 250 (S) to 350 (S) inclusive.

In addition, the changeable range from the lower limit to the upper limit inclusive for the temperature condition of each cold stimulus can be set to a range from 10° C. to 30° C. inclusive, and the changeable range from the lower limit to the upper limit inclusive for the application-period condition of each hot stimulus can be set to a range from 10 (S) to 60 (S) inclusive.

The HCU 40 can include a variable order of priority among the first to fifth personal characteristic items identifiable by the personal characteristic identifier 410, and can include a variable selection number for selecting, from the highest-priority item in the first to fifth personal characteristic items, at least one limited item whose number matches with the variable limit number.

That is, the hot-cold stimulus controller 440 can be configured to

(1) Specify, from the first to fifth personal characteristic items identifiable by the personal characteristic identifier 410, the at least one limited item whose number matches with the variable limit number

(2) Extract at least one default setting value corresponding to the at least one limited item from the table TA0

(3) Extract at least one adjusted value correspond to the at least one limited item from the corresponding at least one of the tables TA1 to TA5

(4) Add the at least one adjusted value to the corresponding at least one default setting value or subtract the at least one adjusted value from the corresponding at least one default setting value, thus changing the corresponding at least one quantitative condition

For example, the table storage 430 can include a priority table TA6 that stores

1. The variable order of priority among the first to fifth personal characteristic items identifiable by the personal characteristic identifier 410

2. The variable limit number for selecting, from the highest-priority item in the first to fifth personal characteristic items, at least one limited item whose number matches with the variable limit number

This configuration enables the hot-cold stimulus controller 440 to refer to the priority table TA6 to thereby specify the at least one limited item in the first to fifth personal characteristic items identifiable by the personal characteristic identifier 410.

This configuration therefore makes it possible to reflect the at least one adjusted value, which corresponds to the at least one limited item having a higher priority than the remaining one or more personal characteristic items, on the corresponding at least one default setting value of hot stimulus and/or cold stimulus. The variable order of priority among the first to fifth personal characteristic items identifiable by the personal characteristic identifier 410 can be preferably set such that, the higher the degree of influence of each of the first to fifth personal characteristic items on the driver's discomfort due to hot stimuli and/or cold stimuli, the higher the priority of the corresponding one of the first to fifth personal characteristic items.

Preferably, the priority of the second personal characteristic item, which represents the information on whether the driver is sensitive to heat or cold, i.e. the driver is hot-natured or cold-natured, or is less-sensitive to any of heat and cold, can be set to the highest, because the degree of influence of the second personal characteristic item on the driver's discomfort due to hot stimuli and/or cold stimuli is the highest in the degrees of influence of all the first to fifth personal characteristic items.

The HCU 40 can be configured to change the order of priority among the first to fifth personal characteristic items stored in the table TA6 according to the current season, i.e. the current time of this year. For example, the processor 40a has a function of measuring the current year and date, so that the processor 40a can change the order of priority among the first to fifth personal characteristic items stored in the table TA6 according to the measured current year and date, i.e. the current season.

The hot-cold stimulus controller 440 therefore can be configured to

(1) Select, from the highest-priority item in the first to fifth personal characteristic items, at least one limited item whose number matches with the variable limit number

(2) Extract at least one default setting value corresponding to the at least one limited item from the table TA0

(3) Extract at least one adjusted value corresponding to the at least one limited item from the corresponding at least one of the tables TA1 to TA5

(4) Add the at least one adjusted value to the corresponding at least one default setting value or subtract the at least one adjusted value from the corresponding at least one default setting value, thus changing the corresponding at least one quantitative condition

For example, the HCU 40 can be configured to change the order of priority among the first to fifth personal characteristic items stored in the table TA6 according to the current season, i.e. the current time of this year, such that

(1) The priority of each of the second and third personal characteristic items is higher than the priorities of the other personal characteristic items upon the current season being winter

(2) The priority of each of the fourth and fifth personal characteristic items is higher than the priorities of the other personal characteristic items upon the current season being summer

Next, the following describes an example of the procedure of the hot-cold stimulus routine carried out by the HCU 40, i.e. the processor 40a of the HCU 40 with reference to FIGS. 7A and 7B.

When starting the hot-cold stimulus routine in response to, for example, when the HCU 40 is switched on, the processor 40a of the HCU 40 serves as, for example, the personal characteristic identifier 410, to identify the first to fifth personal characteristic items, which are simply illustrated in first to fifth items in FIGS. 7A and 7B, of a driver sitting on the driver's seat Se using, for example, the communication module 5, the mobile terminal 2, the camera unit 41, the thermal image measurement device 42, and the operation device 43 set forth above in step S1.

Next, the processor 40a serves as, for example, the sex identifier 411 to determine whether the identified first personal characteristic item represents the driver being female, i.e. the identified sex of the driver being female in step S2. Upon determination that the identified first personal characteristic item represents the driver being female (YES in step S2), the processor 40a serves as, for example, the hot-cold stimulus controller 440 to

(1) Increase the first common setting temperature TE1 of the default hot-cold conditions for each hot stimulus as compared with the first common setting temperature TE1 being used for the driver being male

(2) Increase the first common setting period P1 of the default hot-cold conditions for each hot stimulus as compared with the first common setting period P1 itself being used for the driver being male

(3) Increase the second common setting temperature TE2 of the default hot-cold conditions for each cold stimulus as compared with the second common setting temperature TE2 itself is used for the driver being male

(4) Reduce the second common setting period P2 of the default hot-cold conditions for each cold stimulus as compared with the second common setting period P2 itself being used for the driver being male in step S3

More specifically, in step S3, the processor 40a serves as, for example, the hot-cold stimulus controller 440 to

1. Add the adjusted value of +1° C. to the first common setting temperature TE1 of the default hot-cold conditions included in the hot-cold request instruction for each hot stimulus

2. Add the adjusted value of +5 (S) to the first common setting period P1 of the default hot-cold conditions included in the hot-cold request instruction for each hot stimulus

3. Add the adjusted value of +2° C. to the second common setting temperature TE2 of the default hot-cold conditions included in the hot-cold request instruction for each cold stimulus

4. Subtract the adjusted value of +5 (S) from the second common setting period P2 of the default hot-cold conditions included in the hot-cold request instruction for each cold stimulus in step S3

After completion of the operation in step S3, the hot-cold stimulus routine proceeds to step S4.

Otherwise, upon determination that the identified first personal characteristic item represents the driver being male (NO in step S2), the hot-cold stimulus routine proceeds to step S4.

Note that, if the priority table TA6 has been stored in the memory 40b, the processor 40a performs the operation in step S3 as long as the priority of the first personal characteristic item belongs to the at least one limited item defined in the priority table TA6. That is, the processor 40a performs the following operation in step S4 instead of performing the operation in step S3 if the priority of the first personal characteristic item does not belong to the at least one limited item defined in the priority table TA6.

In step S4, the processor 40a serves as, for example, the heat/cold sensitive identifier 412 to determine whether the identified second personal characteristic item represents the driver being sensitive to heat.

Upon determination that the identified second personal characteristic item represents the driver being sensitive to heat or cold (“sensitive to heat or cold” in step S4), the processor 40a serves as, for example, the hot-cold stimulus controller 440 to

(1) Reduce the first common setting temperature TE1 of the default hot-cold conditions for each hot stimulus as compared with the first common setting temperature TE1 itself being used for the driver being less sensitive to any of heat and cold

(2) Reduce the first common setting period P1 of the default hot-cold conditions for each hot stimulus as compared with the first common setting period P1 itself being used for the driver being less sensitive to any of heat and cold

(3) Reduce the second common setting temperature TE2 of the default hot-cold conditions for each cold stimulus as compared with the second common setting temperature TE2 itself being used for the driver being less sensitive to any of heat and cold

(4) Increase the second common setting period P2 of the default hot-cold conditions for each cold stimulus as compared with the second common setting period P2 itself being used for the driver being less sensitive to any of heat and cold in step S5

More specifically, in step S5, the processor 40a serves as, for example, the hot-cold stimulus controller 440 to

1. Subtract the adjusted value of +3° C. from the first common setting temperature TE1 of the default hot-cold conditions included in the hot-cold request instruction for each hot stimulus

2. Subtract the adjusted value of +10 (S) from the first common setting period P1 of the default hot-cold conditions included in the hot-cold request instruction for each hot stimulus

3. Subtract the adjusted value of +1° C. from the second common setting temperature TE2 of the default hot-cold conditions included in the hot-cold request instruction for each cold stimulus

4. Add the adjusted value of +10 (S) to the second common setting period P2 of the default hot-cold conditions included in the hot-cold request instruction for each cold stimulus

After completion of the operation in step S5, the hot-cold stimulus routine proceeds to step S7

Otherwise, upon determination that the identified second personal characteristic item represents the driver being sensitive to cold (“sensitive to cold” in step S4), the processor 40a serves as, for example, the hot-cold stimulus controller 440 to

(1) Increase the first common setting temperature TE1 of the default hot-cold conditions for each hot stimulus as compared with the first common setting temperature TE1 itself being used for the driver being less sensitive to any of heat and cold

(2) Increase the first common setting period P1 of the default hot-cold conditions for each hot stimulus as compared with the first common setting period P1 itself being used for the driver being less sensitive to any of heat and cold

(3) Increase the second common setting temperature TE2 of the default hot-cold conditions for each cold stimulus as compared with the second common setting temperature TE2 itself being used for the driver being less sensitive to any of heat and cold

(4) Reduce the second common setting period P2 of the default hot-cold conditions for each cold stimulus as compared with the second common setting period P2 itself being used for the driver being less sensitive to any of heat and cold in step S6

More specifically, in step S6, the processor 40a serves as, for example, the hot-cold stimulus controller 440 to

1. Add the adjusted value of +1° C. to the first common setting temperature TE1 of the default hot-cold conditions included in the hot-cold request instruction for each hot stimulus

2. Add the adjusted value of +10 (S) to the first common setting period P1 of the default hot-cold conditions included in the hot-cold request instruction for each hot stimulus

3. Add the adjusted value of +3° C. to the second common setting temperature TE2 of the default hot-cold conditions included in the hot-cold request instruction for each cold stimulus

4. Subtract the adjusted value of +10 (S) from the second common setting period P2 of the default hot-cold conditions included in the hot-cold request instruction for each cold stimulus

After completion of the operation in step S6, the hot-cold stimulus routine proceeds to step S7.

On the other hand, upon determination that the identified second personal characteristic item represents the driver being less-sensitive to any of heat and cold (“less sensitive to heat, cold” in step S4), the hot-cold stimulus routine proceeds to step S7.

Note that, if the priority table TA6 has been stored in the memory 40b, the processor 40a performs the operation in step S5 or S6 as long as the priority of the second personal characteristic item belongs to the at least one limited item defined in the priority table TA6. That is, the processor 40a performs the following operation in step S7 instead of performing the operation in step S5 or S6 if the priority of the second personal characteristic item does not belong to the at least one limited item defined in the priority table TA6.

In step S7, the processor 40a serves as, for example, the poor-circulation identifier 413 to determine whether the identified third personal characteristic item represents the driver having poor circulation. Upon determination that the identified third personal characteristic item represents the driver having poor circulation (YES in step S7), the processor 40a serves as, for example, the poor-circulation identifier 413 to

(1) Increase the second common setting temperature TE2 of the default hot-cold conditions for each cold stimulus to the driver's waist as compared with the second common setting temperature TE2 itself being used for the driver having no poor circulation

(2) Reduce the second common setting period P2 of the default hot-cold conditions for each cold stimulus to the driver's waist as compared with the second common setting period P2 itself being used for the driver having no poor circulation, or

(3) Disable cold stimuli to the driver's waist in step S8

After completion of the operation in step S8, the hot-cold stimulus routine proceeds to step S9.

Otherwise, upon determination that the identified third personal characteristic item represents the driver having no poor circulation (NO in step S7), the hot-cold stimulus routine proceeds to step S9.

Note that, if the priority table TA6 has been stored in the memory 40b, the processor 40a performs the operation in step S8 as long as the priority of the third personal characteristic item belongs to the at least one limited item defined in the priority table TA6. That is, the processor 40a performs the following operation in step S9 instead of performing the operation in step S8 if the priority of the third personal characteristic item does not belong to the at least one limited item defined in the priority table TA6.

In step S9, the processor 40a serves as, for example, the body size identifier 414 to determine whether the identified fourth personal characteristic item represents the driver having a height equal to or more than the predetermined reference height, i.e. 160 centimetres.

Upon determination that the identified fourth personal characteristic item represents the driver having a height equal to or more than the predetermined reference height, i.e. 160 centimetres (YES in step S9), the processor 40a serves as, for example, the hot-cold stimulus controller 440 to

(1) Reduce the first common setting temperature TE1 of the default hot-cold conditions for each hot stimulus as compared with the first common setting temperature TE1 itself being used for the driver whose height being less than the reference height

(2) Reduce the first common setting period P1 of the default hot-cold conditions for each hot stimulus as compared with the first common setting period P1 itself being used for the driver whose height being less than the reference height

(3) Keep the second common setting temperature TE2 of the default hot-cold conditions for each cold stimulus unchanged

(4) Increase the second common setting period P2 of the default hot-cold conditions for each cold stimulus as compared with the second common setting period P2 itself being used for the driver whose height being less than the reference height in step S10

More specifically, in step S10, the processor 40a serves as, for example, the hot-cold stimulus controller 440 to

1. Subtract the adjusted value of +1° C. from the first common setting temperature TE1 of the default hot-cold conditions included in the hot-cold request instruction for each hot stimulus

2. Subtract the adjusted value of +10 (S) from the first common setting period P1 of the default hot-cold conditions included in the hot-cold request instruction for each hot stimulus

3. Keep the second common setting temperature TE2 of the default hot-cold conditions included in the hot-cold request instruction for each cold stimulus

4. Subtract the adjusted value of +5 (S) from the second common setting period P2 of the default hot-cold conditions included in the hot-cold request instruction for each cold stimulus

After completion of the operation in step S10, the hot-cold stimulus routine proceeds to step S11.

On the other hand, upon determination that the identified fourth personal characteristic item represents the driver having a height less than the reference height (NO in step S9), the hot-cold stimulus routine proceeds to step S11.

Note that, if the priority table TA6 has been stored in the memory 40b, the processor 40a performs the operation in step S10 as long as the priority of the fourth personal characteristic item belongs to the at least one limited item defined in the priority table TA6. That is, the processor 40a performs the following operation in step S11 instead of performing the operation in step S10 if the priority of the fourth personal characteristic item does not belong to the at least one limited item defined in the priority table TA6.

In step S11, the processor 40a serves as, for example, the body size identifier 414 to determine whether the identified fifth personal characteristic item represents the driver having a weight equal to or more than the predetermined reference weight, i.e. 80 kilograms.

In step S11, upon determination that the identified fifth personal characteristic item represents the driver having a weight equal to or more than the predetermined reference height, i.e. 80 kilograms (YES in step S11), the processor 40a serves as, for example, the hot-cold stimulus controller 440 to

(1) Reduce the first common setting temperature TE1 of the default hot-cold conditions for each hot stimulus as compared with the first common setting temperature TE1 itself being used for the driver whose height being less than the reference height

(2) Reduce the first common setting period P1 of the default hot-cold conditions for each hot stimulus as compared with the first common setting period P1 itself being used for the driver whose height being less than the reference height

(3) Keep the second common setting temperature TE2 of the default hot-cold conditions for each cold stimulus unchanged

(4) Increase the second common setting period P2 of the default hot-cold conditions for each cold stimulus as compared with the second common setting period P2 itself being used for the driver whose height being less than the reference height in step S12

More specifically, in step S12, the processor 40a serves as, for example, the hot-cold stimulus controller 440 to

1. Subtract the adjusted value of +1° C. from the first common setting temperature TE1 of the default hot-cold conditions included in the hot-cold request instruction for each hot stimulus

2. Subtract the adjusted value of +10 (S) from the first common setting period P1 of the default hot-cold conditions included in the hot-cold request instruction for each hot stimulus

3. Keep the second common setting temperature TE2 of the default hot-cold conditions included in the hot-cold request instruction for each cold stimulus

4. Subtract the adjusted value of +5 (S) from the second common setting period P2 of the default hot-cold conditions included in the hot-cold request instruction for each cold stimulus

After completion of the operation in step S12, the hot-cold stimulus routine proceeds to step S13.

On the other hand, upon determination that the identified fifth personal characteristic item represents the driver having a weight less than the reference weight (NO in step S11), the hot-cold stimulus proceeds to step S13.

Note that, if the priority table TA6 has been stored in the memory 40b, the processor 40a performs the operation in step S10 as long as the priority of the fourth personal characteristic item belongs to the at least one limited item defined in the priority table TA6. That is, the processor 40a performs the following operation in step S11 instead of performing the operation in step S10 if the priority of the fourth personal characteristic item does not belong to the at least one limited item defined in the priority table TA6.

In step S13, the processor 40a sends the hot-cold request instruction including the default hot-cold conditions, which have been changed or unchanged, to the seat ECU 60 of the hot-cold stimulator 6 to thereby instruct the seat ECU 60 to control, based on the hot-cold request instruction, the air mix damper 65 and each of the open/close dampers 66 of the respective vents WD, BD, and SD to thereby repeatedly apply a cycle of alternate hot and cold stimuli to at least one of the stimulus parts of waist, back, and shoulder in synchronization with each other while

(1) The temperature of each hot stimulus is set to the first common setting temperature TE1 or the adjusted temperature

(2) The application period of each hot stimulus is set to the first setting period P1 or the adjusted period

(3) The temperature of each cold stimulus is set to the second common setting temperature TE2 or the adjusted temperature

(4) The application period of each cold stimulus is set to the second setting period P2 or the adjusted period

Thereafter, the processor 40a terminates the hot-cold stimulus routine.

Note that the processor 40a performs the operation in step S2, the operation in step S4, the operation in step S7, the operation in step S9, and the operation in step S11 in this order, but can perform these operations in a free order or parallely perform at least some of them. The processor 40a therefore can perform the operation in step S3, the operation in step S5 or S6, the operation in step S8, the operation in step S10, and the operation in step S12 in a free order or parallely perform at least some of them.

As described above, the operation in step S3 reduces the temperature of each cold stimulus to a female driver and/or reduces the application period of each cold stimulus being applied to the driver being female. This weakens each cold stimulus to the driver being female to thereby reduce the woman's discomfort due to overcooling. The operation in step S3 also causes the temperature of each hot stimulus to the driver being female to be stronger and/or causes the application period of each hot stimulus being applied to the driver being female to be stronger. This prevents overcooling for the driver being female, thus further reducing the woman's discomfort due to overcooling.

Additionally, the operation in step S5 reduces the temperature of each hot stimulus to the driver being sensitive to heat and/or reduces the application period of each hot stimulus being applied to the driver being sensitive to heat. This weakens each hot stimulus to the driver being sensitive to heat. The operation in step S5 also reduces the temperature of each cold stimulus to the driver being sensitive to heat and/or increases the application period of each cold stimulus being applied to the driver being sensitive to heat. This causes each cold stimulus to the driver being sensitive to heat to be higher to thereby prevent overheating for the driver being sensitive to heat. This results in reduction in the discomfort of the driver, who is sensitive to heat, due to overheating.

The operation in step S6 increases the temperature of each cold stimulus to the driver being sensitive to cold and/or reduces the application period of each cold stimulus being applied to the driver being sensitive to cold. This weakens each cold stimulus to the driver being sensitive to cold. The operation in step S6 also increases the temperature of each hot stimulus to the driver being sensitive to cold and/or increases the application period of each hot stimulus being applied to the driver being sensitive to cold. This causes each hot stimulus to the driver being sensitive to cold to be higher to thereby prevent overcooling for the driver being sensitive to cold. This results in reduction in the discomfort of the driver, who is sensitive to cold, due to overcooling.

The operation in step S8 increases the temperature of each cold stimulus to the driver having poor circulation to be higher and/or reduces the application period of each cold stimulus being applied to the driver having poor circulation to be lower. The operation in step S8 can disable the cold stimuli to the driver having poor circulation. This reduces that the driver, who has poor circulation, feels cold, resulting in reduction in the discomfort of the driver, who has poor circulation, due to cooling of the driver. The operation in step S8 can increase the temperature of each hot stimulus to the driver having poor circulation and/or increases the application period of each hot stimulus being applied to the driver having poor circulation. This modification reduces that the driver, who has poor circulation, feels cold, further resulting in reduction in the discomfort of the driver, who has poor circulation, due to overcooling.

Usually, drivers each having a height higher than the reference height, such as 160 centimetres, may be likely to be sensitive to excessive heat. This is because the area that the body of each of the higher drivers covers the driver's seat Se is likely to be larger than each of the drivers having a height lower than the reference height, resulting in heat being likely to persist between the body and the seat Se. From this viewpoint, the operation in step S10 causes each hot stimulus to the higher driver to be weaker and/or causes the temperature each cold stimulus to the higher driver to be stronger to thereby prevent overheating for the higher driver, thus reducing the discomfort of the driver, who is sensitive to heat, due to overheating.

In addition, usually, drivers each having a weight higher than the reference weight, such as 80 kilograms, may be likely to be sensitive to excessive heat. This is because the area that the body of each of the higher drivers covers the driver's seat Se is likely to be larger than each of the drivers having a weight lower than the reference weight, resulting in heat being likely to persist between the body and the seat Se. From this viewpoint, the operation in step S10 reduces each hot stimulus to the higher driver and/or increases each cold stimulus to the higher driver to thereby prevent overheating for the higher driver, thus reducing the discomfort of the driver, who is sensitive to heat, due to overheating.

Note that the HCU 40 is configured to individually identify the height and weight of a driver sitting on the driver's seat Se, but the present disclosure is not limited to this configuration. Specifically, the HCU 40 can be configured to identify the body size, which includes the height and weight, of a driver sitting on the driver's seat Se. In this modification, the HCU 40 and the hot-cold stimulator 6 can be configured to reduce each hot stimulus or increase each cold stimulus to the driver upon determination that at least one of the height and weight of the driver is equal to or more than a corresponding one of the reference height and reference weight.

The HCU 40 and the hot-cold stimulator 6 can be configured to stop application of hot stimuli or cold stimuli to a specified stimulus part of the driver for a corresponding application period upon determination that hot stimulus or cold stimulus is disabled for the specified stimulus part.

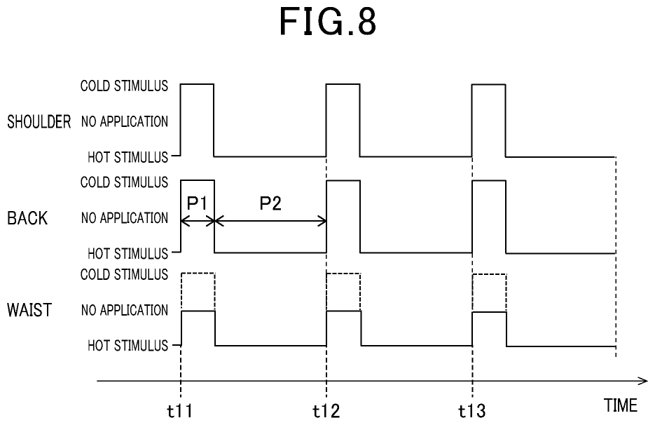

The following describes how the hot-cold stimulator 6 performs the cycle of alternate hot and cold stimuli the predetermined number of times under the condition of disabling cold stimuli to, for example, the waist of a driver sitting on the driver's seat Se with reference to FIG. 8.

As illustrated in FIG. 8 like FIG. 5, the seat ECU 60 of the hot-cold stimulator 6 controls, based on the hot-cold request instruction including disabling of cold stimuli to the waist, the air mix damper 65 and each of the open/close dampers 66 of the respective vents WD, BD, and SD to thereby perform

(1) A first cycle of an application of a hot stimulus to each of the waist, back, and shoulder of the driver for, for example, the first common setting period P1 at time t11, and, thereafter, an application of a cold stimulus to each of the back and shoulder of the driver for, for example, the second common setting period P2 while stopping an application of a cold stimulus to the waist for the same second common setting period P2

(2) A second cycle of an application of a hot stimulus to each of the waist, back, and shoulder of the driver for, for example, the first common setting period P1 at time t12, and, thereafter, an application of a cold stimulus to each of the back and shoulder of the driver for, for example, the second common setting period P2 while stopping an application of a cold stimulus to the waist for the same second common setting period P2

Although above description shows that the hot-cold stimulator 6 performs the cycle of alternate hot and cold stimuli only two times, but the hot-cold stimulator 6 can perform the cycle of alternate hot and cold stimuli the predetermined number of times.

As described above, for each cycle, the hot-cold stimulator 6 stops an application of any of a hot stimulus and a cold stimulus to the waist without making earlier the timing of the next hot stimulus. This configuration makes it possible to keep constant each cycle of alternate hot and cold stimuli while disabling an application of hot stimuli or cold stimuli to a specified part of the driver. This therefore enables the hot-cold stimulator 6 to more simply control each cycle of alternate hot and cold stimuli.

The hot-cold stimulus controller 440 is preferably configured to change, by at least 1° C., the temperature of at least one of the hot stimuli or at least one of the cold stimuli according to the identified personal characteristic items of a driver sitting on the driver's seat Se. This configuration enables the driver to easily feel change in temperature of the hot stimuli or change in temperature of the cold stimuli, making it possible for the driver to be easily conscious of an improvement effect of temperature change of the hot stimuli or the cold stimuli. This therefore further reduces the driver's discomfort.

The hot-cold stimulus controller 440 is preferably configured to individually

(1) Vary, by a variable change width, at least one of the quantitative conditions for hot stimuli

(2) Vary, by a variable change width, at least one of the quantitative conditions for cold stimuli

This configuration makes it possible individually change at least one of the quantitative conditions for hot stimuli and at least one of the quantitative conditions for cold stimuli according to the identified driver's personal characteristic items.

For example, the hot-cold stimulus controller 440 can be configured to greatly reduce at least one of the quantitative conditions for hot stimuli upon the driver being sensitive to heat while maintaining the corresponding at least one of the quantitative conditions for cold stimuli to be smaller. This efficiently prevents overheating of the driver to thereby reduce the driver's discomfort due to heat while preventing overcooling of the driver to thereby reduce the driver's discomfort due to cold.

As another example, the hot-cold stimulus controller 440 can be configured to greatly reduce at least one of the quantitative conditions for cold stimuli upon the driver being female or sensitive to cold while maintaining the corresponding at least one of the quantitative conditions for hot stimuli to be smaller. This efficiently prevents overcooling of the driver to thereby reduce the driver's discomfort due to cold while preventing overheating of the driver to thereby reduce the driver's discomfort due to heat.

The hot-cold stimulus controller 440 can be configured to individually change, by a variable change width, at least one of the quantitative conditions for hot stimuli, and change, by a variable change width, at least one of the quantitative conditions for cold stimuli in accordance with the other driver's personal characteristics except the sex and the heat/cold sensitivities set forth above.

The hot-cold stimulus controller 440 can be configured to

(1) Disable variation of some hot-cold conditions associated with the cold-natured or poor-circulation characteristics upon the insolation determiner 420 determining that solar radiation is hitting the driver

(2) Enable variation of some hot-cold conditions associated with the cold-natured or poor-circulation characteristics upon the insolation determiner 420 determining that solar radiation is not hitting the driver

For example, the hot-cold stimulus controller 440 can be configured to disable

(1) Variation of at least one of the quantitative conditions for weakening each cold stimulus, such as an increase of the temperature of each cold stimulus or reduce the period of each cold stimulus (see, for example, step S6)

(2) Variation of at least one of the quantitative conditions for strengthening each hot stimulus, such as an increase of the temperature of each hot stimulus or increase the period of each hot stimulus (see, for example, step S6)

This configuration prevents an increase in each hot stimulus or a reduction in each cold stimulus to the driver who is being heated by solar radiation, resulting in reduction of the discomfort of the driver due to overheating of the driver. Additionally, this configuration prevents a reduction in each cold stimulus or an increase in each hot stimulus to the driver who is free from solar radiation, resulting in reduction of the discomfort of the driver due to overcooling of the driver.

As described above, the HCU 40 of the fatigue reduction facilitating system 1 according to the first embodiment includes the personal characteristic identifier 410 and the hot-cold stimulus controller 440.

The personal characteristic identifier 410 identifies a plurality of personal characteristic items including

1. The sex of the driver as the first personal characteristic item

2. The information on whether the driver is sensitive to heat or cold is less-sensitive to any of heat and cold as the second personal characteristic item

3. The information on whether the driver has poor circulation as the third personal characteristic item

4. The height of the driver as the fourth personal characteristic item

5. The weight of the driver as the fifth personal characteristic item

The hot-cold stimulus controller 440 varies, based on the identified first to fifth personal characteristic items, at least one of

(1) At least one part of the driver to which each hot stimulus and/or each cold stimulus are to be applied from the hot-cold stimulator 6

(2) The temperature of each hot stimulus and/or each cold stimulus to be applied from the hot-cold stimulator to the at least one part of the driver

(3) The application period of each hot stimulus and/or each cold stimulus to be applied from the hot-cold stimulator to the at least one part of the driver

The first to fifth personal characteristic items of sex, heat/cold sensitive information, poor-circulation information, height, and weight of the driver are estimated to have a high correlation with the sensitivity of the driver to the hot/cold stimuli. For this reason, for each hot stimulus, where the corresponding hot stimulus is applied to, the temperature of the corresponding hot stimulus, and the application period of the corresponding hot stimulus to the driver may lead to an increase of discomfort of the driver having these personal characteristic items. Similarly, for each cold stimulus, where the corresponding cold stimulus is applied to, the temperature of the corresponding cold stimulus, and the application period of the corresponding cold stimulus to the driver may lead to an increase of discomfort of the driver having these personal characteristic items.

From this viewpoint, the HCU 40 of the first embodiment is configured to vary, for each hot-cold stimulus,

(1) At least one part of the driver to which the corresponding hot-cold stimulus are to be applied from the hot-cold stimulator 6 to thereby prevent an increase in the driver's uncomfortable feeling while reducing the driver's fatigue

(2) The temperature of the corresponding hot-cold stimulus to thereby prevent an increase in the driver's uncomfortable feeling while reducing the driver's fatigue

(3) The application period of the corresponding hot-cold stimulus to thereby prevent an increase in the driver's uncomfortable feeling while reducing the driver's fatigue

This configuration therefore makes it possible to reduce, for each driver, the individual uncomfortable feeling due to application of the hot/cold stimuli to the corresponding driver for reducing fatigue of the corresponding driver.

Note that the first embodiment shows an example of how the HCU 40 of the first embodiment changes the hot-cold conditions for each hot-cold stimulus, and therefore the HCU 40 can change the hot-cold conditions for each hot-cold stimulus in a selected one of various modes.

For example, if a driver sitting on the driver's seat Se has the first to fifth personal characteristic items, which show a higher sensitive to cold, the HCU 40 can be configured to

(1) Reduce at least one of the quantitative conditions for each cold stimulus to at least one part of the driver, which is a higher sensitive to cold

(2) Exclude the at least one higher cold-sensitive part to which each hot stimulus is to be applied and/or

(3) Increase at least one of the quantitative conditions for each cold stimulus to the at least one higher cold-sensitive part

Similarly, if a driver sitting on the driver's seat Se has the first to fifth personal characteristic items, which show a higher sensitive to heat, the HCU 40 can be configured to

(1) Reduce at least one of the quantitative conditions for each hot stimulus to at least one part of the driver, which is a higher sensitive to heat

(2) Exclude the at least one higher hot-sensitive part to which each hot stimulus is to be applied and/or

(3) Increase at least one of the quantitative conditions for each cold stimulus to the at least one higher hot-sensitive part

Note that reducing at least one of the quantitative conditions for each cold stimulus is, for example, to increase the default value, i.e. the reference value, of the temperature of each cold stimulus, and/or reduce the default value, i.e. the reference value, of the application period of each cold stimulus. In contrast, increasing at least one of the quantitative conditions for each cold stimulus is, for example, to reduce the default value, i.e. the reference value, of the temperature of each cold stimulus, and/or increase the default value, i.e. the reference value, of the application period of each cold stimulus.

Similarly, reducing at least one of the quantitative conditions for each hot stimulus is, for example, to reduce the default value, i.e. the reference value, of the temperature of each hot stimulus, and/or reduce the default value, i.e. the reference value, of the application period of each hot stimulus. In contrast, increasing at least one of the quantitative conditions for each hot stimulus is, for example, to increase the default value, i.e. the reference value, of the temperature of each hot stimulus, and/or increase the default value, i.e. the reference value, of the application period of each cold stimulus.

### Second Embodiment

The following describes the second embodiment of the present disclosure. The structures and/or functions of a fatigue reduction facilitating system according to the second embodiment are different from those of the fatigue reduction facilitating system according to the first embodiment in the following points. The following therefore mainly describes the different points.

The fatigue reduction facilitating system 1 according to the first embodiment is configured to apply the hot stimuli to each of predetermined stimulus parts, i.e. waist, back, and shoulder, of a driver sitting on the driver's seat Se while the temperature of the hot stimuli is unchanged among the stimulus parts different from each other.

In contrast, the fatigue reduction facilitating system according to the second embodiment is comprised of a predetermined number of the hot-cold stimulators 6 provided for the respective vents WD, BD, and SD (see dashed two-dot line in FIG. 1). That is, each of the hot-cold stimulators 6 is configured to individually apply the hot stimuli and/or cold stimuli to the corresponding one of the waist, back, and shoulder via the respective vents WD, BD, and SD while individually adjusting the temperature of each hot stimulus and/or cold stimulus. This configuration enables the temperatures of the hot stimuli applied to the respective parts to be different from one another, and also enables the temperatures of the cold stimuli applied to the respective parts to be different from one another.

In addition, each of the hot-cold stimulators 6 is configured to individually apply the hot stimuli and/or cold stimuli to the corresponding one of the waist, back, and shoulder via the respective vents WD, BD, and SD while individually adjusting the application period of each hot stimulus and/or cold stimulus. This configuration enables the application periods of the hot stimuli applied to the vents WD, BD, and SD to be different from one another, and also enables the application periods of the cold stimuli applied to the vents WD, BD, and SD to be different from one another.

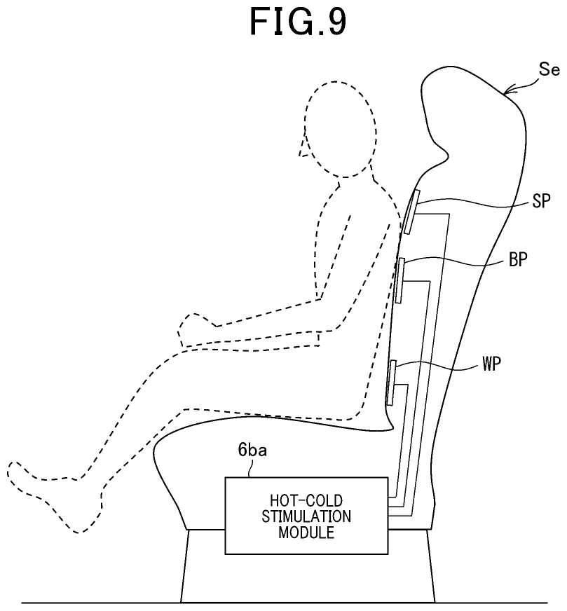

FIG. 9 schematically illustrates a modification of the second embodiment.

A fatigue reduction facilitating system 1A according to the modification of the second embodiment illustrated in FIG. 9. The fatigue reduction facilitating system 1A includes a plurality of thermoelectric devices WP, BP, and SP, such as Peltier-effect pads or heating-wire pads. The thermoelectric devices WP, BP, and SP are disposed in the driver's seat Se and located to be close to the respective waist, back, and shoulder of the driver.

A hot-cold stimulation module 6ab is electrically connected to the thermoelectric devices WP, BP, and SP. The hot-cold stimulation module 6a controls how to apply a voltage to at least one pad selected from the thermoelectric devices WP, BP, and SP to thereby switch heating or cooling the corresponding at least one part, thus applying hot stimuli and/or cold stimuli to the at least one selected part of the driver.

In particular, the hot-cold stimulation module 6a can control the magnitude and period of a voltage to be applied to at least one pad selected from the thermoelectric devices WP, BP, and SP to thereby adjust the temperature of each hot or cold stimulus to a selected at least one part of the driver.

The fatigue reduction facilitating system 1A according to the modification of the second embodiment can be comprised of blowers in addition to the thermoelectric devices WP, BP, and SP. That is, the fatigue reduction facilitating system 1A can be configured such that each blower blows out air, which is heated by a corresponding one of the thermoelectric devices WP, BP, and SP, to a corresponding one of the parts of the driver to thereby apply a heat stimulus thereto, and blows out air, which is cooled by a corresponding one of the thermoelectric devices WP, BP, and SP, to a corresponding one of the parts of the driver to thereby apply a cold stimulus thereto.

As described above, each of the fatigue reduction facilitating systems according to the second embodiment is configured to

1. Apply the hot stimuli to the respective parts of the driver while changing the temperatures of the hot stimuli to the respective parts to be different from each other

2. Apply the cold stimuli to the respective parts of the driver while changing the temperatures of the hot stimuli to the respective parts to be different from each other

This configuration makes it possible to apply, to each of the waist, back, and shoulder of the driver, each hot stimulus and/or each cold stimulus while individually setting the temperature of the corresponding hot stimulus and/or cold stimulus to be a value suitable for the corresponding one of the waist, back, and shoulder.

For example, this configuration can prevent the temperature of each cold stimulus to be applied to the waist and shoulder, which are easily to be stiff, from excessively decreasing while reducing the temperature of each cold stimulus to be lower than the temperature of each cold stimulus to the waist or shoulder. This prevents the driver from feeling discomfort due to overheating.

### Third Embodiment

The following describes the third embodiment of the present disclosure.

The personal characteristic identifier 410 according to the first embodiment is configured to identify the first to fifth personal characteristic items of sex, heat/cold sensitive information, poor-circulation information, height, and weight of a driver sitting on the driver's seat Se, but the present disclosure is not limited to the configuration.

Specifically, the personal characteristic identifier 410 according to the third embodiment is configured to identify a part of the first to fifth personal characteristic items of sex, heat/cold sensitive information, poor-circulation information, height, and weight of a driver sitting on the driver's seat Se, or identify an additional personal characteristic item.

### Fourth Embodiment

The following describes the fourth embodiment of the present disclosure.

The hot-cold stimulator 6 of the first embodiment is configured to repeat the cycle of the alternate hot and cold stimuli to a selected part of the driver a predetermined number of times, but the present disclosure is not limited to this configuration.

Specifically, the hot-cold stimulator 6 of the fourth embodiment is configured to apply, for each part of the driver, both of or one of the hot stimuli and cold stimuli one or more times. For example, the hot-cold stimulator 6 of the fourth embodiment can be configured to apply only the hot stimuli to each of the waist and shoulder of the driver, and apply only the cold stimuli to the back of the driver.

### Fifth Embodiment

The following describes the fifth embodiment of the present disclosure.

The hot-cold stimulator 6 of the first embodiment is configured to apply the hot and cold stimuli to each of the waist, back, and shoulder of the driver as an example, but the present disclosure is not limited to this configuration.

Specifically, the hot-cold stimulator 6 of the fifth embodiment is configured to apply the hot and cold stimuli to a part of the waist, back, and shoulder of the driver, or apply the hot and cold stimuli to another part of the driver in addition to the waist, back, and shoulder of the driver.

### Sixth Embodiment

The following describes the sixth embodiment of the present disclosure.

The hot-cold stimulator 6 of the first embodiment is installed in the driver's seat Se of the vehicle V, but the present disclosure is not limited to this configuration.

Specifically, the hot-cold stimulator 6 of a fatigue reduction facilitating system according to the sixth embodiment is installed in an occupant seat except for the driver's seat Se for reducing fatigue of an occupant sitting on the occupant seat.

The hot-cold stimulator 6 of a fatigue reduction facilitating system according to a modification of the sixth embodiment can be installed in each of the driver's seat Se and at least one occupant seat.

In this modification, the fatigue reduction facilitating system can be configured such that the camera unit 41 and the thermal image measurement device 42 are provided in the interior of the vehicle V for each of the driver's seat Se and the at least one occupant seat.

Specifically, the camera unit 41 provided for driver's seat Se is configured to periodically capture the driver images, and the camera unit 41, and the camera unit 41 provided for the at least one occupant seat is configured to periodically capture images of at least one occupant sitting on the at least one occupant seat. The

The thermal image measurement device 42 provided for driver's seat Se is configured to measure the temperature-distribution image of the driver sitting on the driver's seat Se, and the thermal image measurement device 42 provided for the at least one occupant seat is configured to measure the temperature-distribution image of the at least one occupant sitting on the at least one occupant seat.

The HCU 40 can be configured to identify the personal characteristic items of the driver in accordance with the driver images and the temperature-distribution image of the driver, and also identify the personal characteristic items of the at least one occupant in accordance with the images of the at least one occupant and the temperature-distribution image of the at least one occupant.

The fatigue reduction facilitating system can be configured such that, independently of the camera unit 41 and the thermal image measurement device 42 being provided for each of the driver's seat Se and the at least one occupant seat, the communication module 5 and/or the operation device 43 are provided for each of the driver's seat Se and the at least one occupant seat. That is, the communication module 5, the operation device 43, and/or the mobile terminal 2 for each of the driver and the at least one occupant can be configured to input, to the HCU 40, various items of information associated with the personal characteristic items of the corresponding one of the driver and the at least one occupant. The HCU 40 can be configured to identify the personal characteristic items of the driver in accordance with the various items of information associated with the personal characteristic items of the driver, and also identify the personal characteristic items of the at least one occupant in accordance with the various items of information associated with the personal characteristic items of the at least one occupant.

The HCU 40 can be provided for each of the driver's seat Se and the at least one occupant seat, or commonly provided for the driver's seat Se and the at least one occupant seat.

Each of the fatigue reduction facilitating systems 1 and 1A according to the above embodiments is used in the automobile V, but the present disclosure is not limited thereto.

Specifically, each of the fatigue reduction facilitating systems 1 and 1A can be used for reducing fatigue of users existing in various mobile objects, for example, used for users in rail vehicles, aircrafts, ships except for automobiles.

Each of the fatigue reduction facilitating systems 1 and 1A can also be used for reducing fatigue of uses existing in house's rooms or facility's rooms.

While illustrative embodiments of the present disclosure have been described herein, the present disclosure is not limited to the embodiments described herein, but includes any and all embodiments having modifications, omissions, combinations (e.g., of aspects across various embodiments), adaptations and/or alternations as would be appreciated by those in the art based on the present disclosure. The limitations in the claims are to be interpreted broadly based on the language employed in the claims and not limited to examples described in the present specification or during the prosecution of the application, which examples are to be construed as non-exclusive.

What is claimed is:

1. An apparatus for facilitating fatigue reduction of a user, the apparatus comprising:
a characteristic identifier configured to identify at least one personal characteristic item of the user; and
a hot-cold stimulus controller configured to:
instruct a hot-cold stimulator to apply a hot stimulus and a cold stimulus to the user in accordance with a stimulus condition; and
vary, based on the identified at least one personal characteristic item of the user, the stimulus condition of each of the hot stimulus and cold stimulus,
the stimulus condition of each of the hot stimulus and cold stimulus including at least one of:
one or more parts of the user to which the corresponding one of the hot stimulus and cold stimulus is to be applied;
a temperature of the corresponding one of the hot stimulus and cold stimulus; and
an application period of the corresponding one of the hot stimulus and cold stimulus to the user.

2. The apparatus according to claim 1, wherein:
the hot-cold stimulus controller is configured to alternately apply the hot stimulus and cold stimulus to a selected part among the one or more parts of the user.

3. The apparatus according to claim 1, wherein:
the at least one personal characteristic item of the user includes at least:
a sex of the user;
heat/cold sensitive information on whether the user is sensitive to heat or cold or is less-sensitive to any of heat and cold;
poor circulation information on whether the user has poor circulation;
a height of the user; and
a weight of the user.

4. The apparatus according to claim 3, wherein:
the at least one personal characteristic item of the user includes the sex of the user; and
the hot-cold stimulus controller is configured to vary at least one of the temperature and application period of the cold stimulus to the user to thereby cause the cold stimulus to be weaker upon the sex of the user being identified as female as compared with the cold stimulus to the user whose sex is identified as male.

5. The apparatus according to claim 4, wherein:
the at least one personal characteristic item of the user includes the sex of the user; and
the hot-cold stimulus controller is configured to vary at least one of the temperature and application period of the hot stimulus to the user to thereby cause the hot stimulus to be stronger upon the sex of the user being identified as female as compared with the hot stimulus to the user whose sex is male.

6. The apparatus according to claim 3, wherein:
the at least one personal characteristic item of the user includes at least one of the height and weight of the user;
the characteristic identifier is configured to identify whether the at least one of the height and weight is equal to or more than a corresponding at least one reference value; and
the hot-cold stimulus controller is configured to vary at least one of the temperature and application period of the hot stimulus to the user to thereby cause the hot stimulus to be weaker upon the at least one of the height and weight being identified to be equal to or more than the corresponding at least one reference value as compared with the hot stimulus to the user whose at least one of the height and weight is less than the corresponding at least one reference value.

7. The apparatus according to claim 3, wherein:
the at least one personal characteristic item of the user includes the information on whether the user is sensitive to heat or cold or is less-sensitive to any of heat and cold; and
the hot-cold stimulus controller is configured to:
reduce at least the temperature of each of the hot stimulus and cold stimulus to the user upon the heat/cold sensitive information representing the user being identified to be sensitive to heat as compared with the user whose heat/cold sensitive information identifies the user as less sensitive to any of heat and cold; and
increase at least the temperature of each of the hot stimulus and cold stimulus to the user upon the heat/cold sensitive information representing the user being identified to be sensitive to cold as compared with the user whose heat/cold sensitive information being identified to be less sensitive to any of heat and cold.

8. The apparatus according to claim 3, wherein:
the at least one personal characteristic item of the user includes poor circulation information on whether the user has poor circulation; and
the hot-cold stimulus controller is configured to vary at least one of the temperature and application period of the cold stimulus to a waist of the user to thereby cause the cold stimulus to be weaker upon the poor circulation information representing the user having poor circulation as compared with the cold stimulus to the user whose poor circulation information is identified not to have poor circulation.

9. The apparatus according to claim 3, further comprising:
an isolation determiner configured to determine whether solar radiation is hitting the user,
wherein:
the at least one personal characteristic item of the user includes the information on whether the user is sensitive to heat or cold or is less-sensitive to any of heat and cold; and
the hot-cold stimulus controller is configured to:
enable variation of the stimulus condition of each of the hot stimulus and cold stimulus upon determination that solar radiation is not hitting the user; and
disable variation of the stimulus condition of each of the hot stimulus and cold stimulus upon determination that solar radiation is hitting the user.

10. The apparatus according to claim 1, wherein:
the stimulus condition of each of the hot stimulus and cold stimulus includes, as a quantitative condition of the corresponding one of the hot stimulus and cold stimulus, at least one of the temperature and application period of the corresponding one of the hot stimulus and cold stimulus to the user; and
the hot-cold stimulus controller is configured to:
store a reference value of the quantitative condition previously determined for the at least one personal characteristic item of the user;
store a variation value for the quantitative condition previously determined for the at least one personal characteristic item of the user; and
perform one of:
adding, for the quantitative condition, the variation value to the reference value to thereby vary the quantitative condition; and
subtracting, for the quantitative condition, the variation value from the reference value to thereby vary the quantitative condition.

11. The apparatus according to claim 10, wherein:
the at least one personal characteristic item of the user includes plural characteristic items of the user; and
the hot-cold stimulus controller is configured to:
store the reference value of the quantitative condition previously determined for each of the plural characteristic items of the user;
store the variation value for the quantitative condition previously determined for each of the plural characteristic items of the user; and
perform one of:
adding, for the quantitative condition, the variation value of each of the plural characteristic items, the reference value of the corresponding one of the plural characteristic items to thereby vary the quantitative condition; and
subtracting, for the quantitative condition, the variation value of each of the plural characteristic items, from the reference value of the corresponding one of the plural characteristic items to thereby vary the quantitative condition.

12. The apparatus according to claim 11, wherein:
the plural characteristic items of the user have a variable order of priority thereamong; and
the hot-cold stimulus controller is configured to:
select a predetermined number of items in the plural characteristic items from a highest priority in accordance with the order of priority; and
perform one of:
adding, for the quantitative condition, the variation value of each of the selected items, the reference value of the corresponding one of the selected items to thereby vary the quantitative condition; and
subtracting, for the quantitative condition, the variation value of each of the selected items, from the reference value of the corresponding one of the selected items to thereby vary the quantitative condition.

13. The apparatus according to claim 12, wherein:
the plural characteristic items of the user include the hot/cold sensitive information; and
the order of priority is determined such that the hot/cold sensitive information has the highest order in all the plural characteristic items.

14. The apparatus according to claim 12, wherein:
the order of priority of the plural characteristic items of the user is determined for each of seasons; and
the hot-cold stimulus controller is configured to:
select one of the orders of priority determined for the respective seasons, the selected one of the orders corresponding to a current season;
select a predetermined number of items in the plural characteristic items from a highest priority in accordance with the selected order of priority; and
perform one of:
adding, for the quantitative condition, the variation value of each of the selected items, the reference value of the corresponding one of the selected items to thereby vary the quantitative condition; and
subtracting, for the quantitative condition, the variation value of each of the selected items, from the reference value of the corresponding one of the selected items to thereby vary the quantitative condition.

15. The apparatus according to claim 10, wherein:
the quantitative condition includes a predetermined changeable range from an upper limit to a lower limit inclusive, the changeable range being set to be narrower than a settable range of the quantitative condition by the hot-cold stimulus controller; and
the hot-cold stimulus controller is configured to vary the quantitative condition within the changeable range from the upper limit to the lower limit inclusive.

16. The apparatus according to claim 1, wherein:
the hot-cold stimulus controller is configured to:
individually vary at least one of the temperature and the application period of the hot stimulus as a first quantitative condition by a first changeable width; and
individually vary at least one of the temperature and the application period of the cold stimulus as a second quantitative condition by a second changeable width independently.

17. The apparatus according to claim 1, wherein:
the hot-cold stimulus controller is configured to vary at least:
the part of the user to which each of the hot stimulus and cold stimulus is to be applied; and
the temperature of each of the hot stimulus and cold stimulus for each of the varied parts of the user.

18. The apparatus according to claim 1, wherein:
the one or more parts of the user to which each of the hot stimulus and cold stimulus is to be applied are plural parts
the hot-cold stimulus controller is configured to:
variably select one of the plural parts of the user to which each of the hot stimulus and cold stimulus is to be applied; and
exclude one of the plural parts of the user to which each of the hot stimulus and cold stimulus is to be applied; and
stop an application of each of the hot stimulus and cold stimulus to the excluded part for the corresponding application period.

19. The apparatus according to claim 1, wherein:
the hot-cold stimulus controller is configured to vary, by at least one degree, the temperature of each of the hot stimulus and cold stimulus for each of the varied parts of the user.

20. A method of facilitating fatigue reduction of a user, the method comprising:
identifying at least one personal characteristic item of the user; and
instructing a hot-cold stimulator to apply a hot stimulus and a cold stimulus to the user in accordance with a stimulus condition; and
varying, based on the identified at least one personal characteristic item of the user, the stimulus condition of each of the hot stimulus and cold stimulus,
the stimulus condition of each of the hot stimulus and cold stimulus including at least one of:
one or more parts of the user to which the corresponding one of the hot stimulus and cold stimulus is to be applied;
a temperature of the corresponding one of the hot stimulus and cold stimulus; and
an application period of the corresponding one of the hot stimulus and cold stimulus to the user.

21. A computer-readable storage medium comprising a set of computer program instructions, the instructions causing a computer to carry out:
a first step of identifying at least one personal characteristic item of the user;
a second step of instructing a hot-cold stimulator to apply a hot stimulus and a cold stimulus to the user in accordance with a stimulus condition; and
a third step of varying, based on the identified at least one personal characteristic item of the user, the stimulus condition of each of the hot stimulus and cold stimulus,
the stimulus condition of each of the hot stimulus and cold stimulus including at least one of:
one or more parts of the user to which the corresponding one of the hot stimulus and cold stimulus is to be applied;
a temperature of the corresponding one of the hot stimulus and cold stimulus; and
an application period of the corresponding one of the hot stimulus and cold stimulus to the user.
